(19) United States
(12) Patent Application Publication
(10) Pub. No.: US 2017/0106101 A1
(43) Pub. Date: Apr. 20, 2017

(54) METHODS INVOLVING MACROPHAGE TUMOR CELL FUSION HYBRIDS

(71) Applicants: Melissa Hirose Wong, Lake Oswego, OR (US); Charles E. Gast, Portland, OR (US)

(72) Inventors: Melissa Hirose Wong, Lake Oswego, OR (US); Charles E. Gast, Portland, OR (US)

(73) Assignee: OREGON HEALTH & SCIENCE UNIVERSITY, Portland, OR (US)

(21) Appl. No.: 15/295,845

(22) Filed: Oct. 17, 2016

Related U.S. Application Data
(60) Provisional application No. 62/242,647, filed on Oct. 16, 2015.

(51) Int. Cl. A61K 49/00; G01N 33/58; G01N 33/574; G01N 33/50; A01K 67/027

(52) U.S. Cl. CPC A61K 49/0008; G01N 33/5011; A01K 67/0271; G01N 33/57438; G01N 33/582; A01K 2207/12; A01K 2217/15; A01K 2227/105; A01K 2267/0331

(57) ABSTRACT

Methods of identifying circulating macrophage-tumor cell fusion hybrids from a subject, methods of screening test compounds for inhibition of activity of in vitro and in vivo derived macrophage-tumor cell fusion hybrids, and methods of assessing the probability of survival of a human subject with pancreatic cancer past one year are disclosed.

### PRIORITY CLAIM

This application claims the benefit of U.S. 62/242,647, filed 16 Oct. 2015, which is entitled METHODS OF IDENTIFYING, COUNTING, AND PURIFYING CIRCULATING TUMOR CELLS and is hereby incorporated by reference in its entirety.

### STATEMENT REGARDING FEDERALLY SPONSORED RESEARCH OR DEVELOPMENT

This invention was made in part using funds from federal grant numbers CA172334 and CA118235 from the National Institutes of Health. Therefore, the United States government has certain rights in the invention.

### FIELD

Generally, the field involves methods of isolating cells, screening methods for active compounds, and diagnostic tests. More specifically, the field involves methods of isolating circulating tumor cells that express macrophage surface markers, screening methods for active compounds that inhibit the activity of circulating tumor cell fusion hybrids, and diagnostic tests involving circulating tumor cells that express macrophage surface markers.

### BACKGROUND

Metastatic cancer fails to respond to effective therapies utilized for primary tumors and therefore accounts for the vast majority of cancer-related deaths. (Nguyen et al, Nat Rev Cancer 9, 274-284 (2009); Talmadge et al, Cancer Res 70, 5649-5669 (2010); Coghlin & Murray, J Pathol 222, 1-15 (2010); National Cancer Institute http://www.cancer.gov/about-cancer, last accessed 12 Oct. 2016; all of which are incorporated by reference herein). It is generally accepted that primary tumor cells acquire behaviors that permit them to escape the primary tumor site, navigate circulation, and colonize in a metastatic site (Fidler, Nat Rev Cancer 3, 453-458 (2003) and Gupta & Massague, Cell 127, 679-695 (2006); both of which are incorporated by reference herein), but the underlying mechanism for this is not fully understood (Nguyen 2009 supra; Talmadge 2010 supra, and Hanahan & Coussens, Cancer Cell 21, 309-322 (2012); incorporated by reference herein). Further, research is only beginning to define how, during the act of metastasis, cancer cells gain heterogeneous properties that complicate treatment effectiveness for late-stage cancer (Heppner, Cancer Res 44, 2259-2265 (1984) and Marusyk & Polyak, Biochim Biophys Acta 1805, 105-117 (2010); both of which are incorporated by reference herein). Clearly, acquired mutations and altered epigenetic regulation rank as top mechanistic candidates contributing to metastatic behaviors, but this knowledge has not yet resulted in development of reliably effective therapeutics (Hanahan 2012 supra). This may be due to the complex nature of tumors and the myriad of undiscovered microenvironmental influences that shape tumor behavior and the acquired heterogeneity that occurs as the tumor progresses.

One possible microenvironmental impact on tumorigenesis concerns macrophage-cancer cell fusion. Cell fusion between immune and cancer cells is a century-old hypothesis (Carter, J Natl Cancer Inst 100, 1279-1281 (2008) and Pawelek, Lancet Oncol 6, 988-993 (2005); both of which are incorporated by reference herein) that has been circumstantially implicated (Pawalek 2005 supra, Powell et al, Cancer Res 71, 1497-1505 (2011); and Lorico et al, Biomed Res Inst 2015 289567 (2015); all of which are incorporated by reference herein), but not definitely demonstrated to functionally affect tumor cell behaviors. Early in vitro studies revealed that cell fusion hybrids harbored more rapid cell cycling times when compared to either of their parental cell lines (Islam et al, Stem Cells and Development 15, 905-919 (2006) and Xue et al, BMC Cancer 15, 793, (2015); both of which are incorporated by reference herein). Further, metastatic cancer cells expressing genes from immune macrophages (Mφs) (Lorico 2015 supra) were highlighted as evidence that aggressive metastatic cells resulted from fusion. Reports of cells that contained components of both immune cells and cancer cells have been increasingly frequent (Patsialou et al, Oncogene 34, 2721-2731 (2015); Orsolya et al, Cancer Res 75 (14 Suppl) Abstract PR06 (2015); Sheng et al, Lab On a Chip 14, 89-98 (2014); and Clawson et al, PLoS One 7, e41052 (2012); all of which are incorporated by reference herein) but there is a lack of definitive evidence for physiologic relevance of these fusion hybrids in neoplastic disease.

### SUMMARY

Disclosed herein are methods of identifying circulating macrophage-tumor cell fusion hybrids from a subject. The methods involve contacting a cell from the blood of the subject with an antibody that binds a macrophage marker. The antibody is conjugated to a first label. The methods further involve contacting the cell with a second antibody that binds a tumor antigen. This antibody is conjugated to a second label distinguishable from the first label. A cell with detectable expression of both the first label and the second label is identified as a circulating macrophage-tumor cell fusion hybrid. In some examples, the antibody that binds the macrophage marker binds CD45. In other examples, the antibody that binds the tumor cell marker binds a cytokeratin. Such antibodies include antibodies that bind individual cytokeratins and pan-cytokeratin antibodies.

In still further examples, the first label includes a fluorescent label that emits at a first wavelength and the second label includes a fluorescent label that emits at a second wavelength such that the first and second wavelength are distinguishable. In such examples the methods can further involve isolating the circulating macrophage tumor cell fusion by fluorescence activated cell sorting (FACS).

Disclosed herein are methods of screening a test compound for inhibition of one or more activities of an in vitro derived macrophage-tumor cell fusion hybrid. The methods involve contacting a sample including an in vitro derived macrophage-tumor cell fusion hybrid with the test compound and contacting another sample including an in vitro derived macrophage-tumor cell fusion hybrid with a negative control. The method further involves measuring one or more of cellular proliferation, cellular migration, or metastasis in the first sample and the second sample. A result showing less cellular proliferation, cellular migration and/or metastasis in the first sample relative to the second sample is an indication that the test compound inhibits one or more activities of an in vitro derived macrophage-tumor cell fusion hybrid.

The methods can further involve generating the in vitro derived macrophage-tumor cell fusion hybrid by contacting a macrophage with a tumor cell in a cell culture lacking an agent that promotes cell-cell fusion. The macrophage expresses a first marker protein and the tumor cell expresses a second marker protein distinguishable from the first marker protein. The methods further involve purifying the macrophage-tumor cell fusion hybrid based on the expression of the first marker protein and the second marker protein. In some examples, the first marker protein includes a first fluorescent protein that fluoresces at a first wavelength, the second marker protein includes a second fluorescent protein that fluoresces at a second wavelength distinguishable from the first wavelength and purifying the macrophage-tumor cell fusion hybrid includes flow cytometry. In these examples, the first fluorescent protein includes RFP, GFP, or YFP and the second fluorescent protein includes RFP, GFP, or YFP, provided that the first and second fluorescent protein are not both RFP, GFP, or YFP.

In still further examples of these methods, measuring proliferation can include an MTS assay. Measuring cellular migration can include a chemotaxis or scratch wound assay, and where measuring metastasis includes injection of the macrophage-tumor cell fusion hybrid into the spleen of an experimental animal and measuring liver metastases. In these examples, both the macrophage and the tumor cell can be derived from a mouse, including a transgenic mouse that expresses a fluorescent protein in macrophages. In still further examples, the tumor cell is derived from a colon adenocarcinoma or melanoma, such as the MC38 or B16F10 cell lines.

Disclosed herein are methods of screening a test compound for inhibition of proliferation or metastasis of an in vivo derived macrophage-melanoma cell fusion hybrid or an in vivo derived mammary tumor cell fusion hybrid. These methods involve injecting a melanoma cell line or a primary mammary tumor into a first mouse. The melanoma cell line expresses a first fluorescent protein. The first mouse transgenically expresses a second fluorescent protein distinguishable from the first fluorescent protein. The melanoma cell line can expand to form a tumor in the first mouse. Expression of the second fluorescent protein in the first mouse occurs at least in macrophages. The method further involves removing the tumor from the first mouse, and purifying cells that express both the first fluorescent protein and the second fluorescent protein by flow cytometry. Cells that express both the first fluorescent protein and the second fluorescent protein are then injected into a second mouse and a third mouse. The test compound is administered to the second mouse and a negative control is administered to the third mouse. A result showing less proliferation and/or metastasis of the cells that express both the first fluorescent protein and the second fluorescent protein in the second mouse relative to the third mouse is an indication that the test compound inhibits proliferation or metastasis of an in vivo derived macrophage-melanoma cell fusion hybrid.

In further examples of the method, the second mouse and the third mouse are each injected with 50-10,000 cells that express the first fluorescent protein and the second fluorescent protein, including 100 cells (50-200 cells) and 3000 cells (2000-4000 cells).

Also disclosed are methods of assessing the chance of survival of a human subject with pancreatic cancer. The method involves receiving a peripheral blood sample from the subject. The sample includes mononuclear cells. The method further involves contacting the blood sample with an anti-CD45 antibody that is conjugated to a first fluorescent label. The blood sample is also contacted with an anti-pan-cytokeratin antibody is conjugated to a second fluorescent label that is distinguishable from the first fluorescent label. The blood sample is also contacted with Hoescht stain. The method further involves counting cells positive for CD45, cytokeratin,and Hoescht stain in at least a subset of the cells in the sample and calculating the percentage of cells positive for CD45, cytokeratin, and Hoescht in the total cells of the subset. If more than 0.8% of the cells in the subset are positive for CD45, cytokeratin and Hoescht, then that indicates that the subject has a less than 12% chance of survival past one year.

In other examples, the methods involve adhering the blood sample to a glass substrate and fixing the cells in 4% paraformaldehyde prior to contacting the blood sample with the first antibody and the second antibody. In still other examples, the peripheral blood sample is a buffy coat fraction. In still other examples, the anti-CD45 antigen is a monoclonal antibody derived from the H130 clone. In still other examples, the anti-pan-cytokeratin antibody is a monoclonal antibody derived from the C1-11 clone or is a polyclonal antiserum. In still other examples, the subset of cells includes at least 2000 cells.

### BRIEF DESCRIPTION OF THE DRAWINGS

Original figures were presented in color. Applicants consider such figures to be part of the original disclosure and reserve the right to present color versions of the originally filed figures in later proceedings.

FIG. 1. Cell fusion in human tumors: Solid tumors from women with previous sex-mismatched bone marrow transplantation (BMT) permits analysis of cell fusion. Box a is an image of pancreatic ductal adenocarcinoma tumor section with cytokeratin (gray), the Y-chromosome (Ychr, red) and Hoechst (blue) detection revealed areas of cytokeratin-positive cells with Y-chromosome-positive nuclei, white arrowhead. The representative areas boxed enlarged in boxes b, c, d, and e. Bar=25 μm.

FIG. 2A is a diagram and set of 3 images showing MC38 (H2B-RFP) cancer cells co-cultured with GFP-expressing McDs result in hybrid cells with RFP nuclei and GFP-expressing cytoplasm (yellow arrowhead) among unfused cancer cells (white arrow) and macrophages (white arrowhead). FIG. 2B is a diagram and set of 3 images showing MC38 (H2B-RFP/Cre) cancer cells co-cultured with McDs expressing the cre reporter, R26R-stop-YFP results in YFP-expressing hybrid cells (yellow arrowhead). FIG. 2C is a set of three flow cytometry plots and an image of an immunoblot showing YFP-expressing hybrids can be FACS-isolated to purify YFP-expressing hybrid cells confirmed by Immunoblot. FIG. 2D is a diagram and set of 8 images showing co-cultured McDs labeled with EdU (green) and MC38 (H2B-RFP/Cre) cancer cells produce YFP-expressing hybrids that initially harbor two nuclei—one from each parent, but upon mitotic division undergo nuclear fusion resulting in a single nucleus with EdU-labeled and RFP-expressing DNA. Hybrid cell outlined in yellow. Bar=10 μm.

FIG. 3A is a plot showing proliferative analyses of MC38 cells and MC38-derived hybrids injected into the flank of an immune competent mouse. FIG. 3B is a plot showing analyses of metastatic seeding of hybrids and MC38 cells injected into the spleen and analyzed in the liver. FIG. 3C is a GoChord display of key metastatic GO pathways and selected differentially regulated genes from the significantly enriched in hybrid versus MC38 comparison (Log2 fold-change). Outer ring denotes hybrid-MC38 and inner ring represents MD-MC38 comparisons. FIG. 3D is an image of gelatin zymography comparing Mmp2 activity in MC38, five different MC38-derived hybrid isolates, and MDs. FIG. 3E is a static portrayal of migration tracks from unfused MC38s (black) and a MC38-derived fusion hybrid (red) generated from live-imaged co-cultures. Mean speed of hybrids (red bar in plot) relative to MC38s (gray bar in plot) is statistically significant, *p<1.1×10⁻⁹. FIG. 3F is a plot of the results of chemotaxis assays towards Csf1 and Sdf1 ligands. Hybrid chemotaxis towards Csf1 and Sdf1 is statistically significant relative to unfused MC38 cells after 24 h (p<0.05). FIG. 3G is a graph showing the results of incubation of cells with antibodies to Csf1 R and Cxcr4 reduce migration of hybrids towards their ligand. p<0.05 and 0.01 respectively. (Hybrid=red bar, MC38=gray bar).

FIG. 4A is a diagram showing the process of B16F10 (H2B-RFP) cells intradermally injected into GFP-expressing mice were harvested at 1 cm of growth. FIG. 4B is a set of images showing fluoresence analyses of tumor for RFP (red) and GFP (green) reveal double-positive hybrids and phagocytosed cancer cells with different nuclear morphology. Bar=25 μm. FIG. 4C is a diagram showing B16F10 (H2B-RFP/Cre) cells injected into R26R-stop-YFP transgenic mice. FIG. 4D is a flow cytometry plot showing hybrid and unfused cancer cells from a dissociated tumor subjected to FACS—hybrids (upper box) and unfused (lower box) cancer cells. FIG. 4E is a graph of the results where 100 FACS-isolated cells injected into wildtype secondary recipient mice analyzed for tumor growth at 40 days. FIG. 4F is a plot of the results where 3,000 FACS-isolated cells injected into recipient mice and temporally monitored for growth. FIG. 4G is a set of seven images showing: primary tumor (subcutaneous view, yellow arrowhead) and metastatic lymph node (LN) growth of hybrid-derived-primary tumor. Both primary tumor and lymph nodes express RFP hybrid cells (white arrowhead) by fluorescence and PCR. Bar in gross=5 mm, Bar in tissue section=50 μm.

FIG. 5A is a diagram of B16F10 (H2B-RFP) cells intradermally injected into a GFP-expressing mouse. FIG. 5B is a flow cytometry plot and a set of three images showing blood collected at time of tumor resection, analyzed by flow cytometry for GFP and RFP expression. RFP+GFP+cells were detectible in pre-sorted cell preparations by immunofluorescence. FIG. 5C is a plot of the percentages of fusion hybrids (RFP+/GFP+) and unfused CTCs (RFP+/GFP−) expressing the leukocyte antigen CD45, * p<0.000002. FIG. 5D is a set of images illustrating Human pancreatic cancer patient peripheral blood analyzed for Cytokeratin+ (green) and CD45+ (red) expression using in situ analyses and digital scanning. FIG. 5E is a plot showing CK+/CD45+ and CK+/CD45− cells quantified in patient blood across cancer stage, *ANOVA p<0.023. FIG. 5F is a Kaplan-Meier Curve of dichotomized biomarkers (fCTC and CTC) showing a statistically significant increased risk of death for fCTC (p=0.0029). FIG. 5G is a Kaplan-Meier Curve of dichotomized biomarkers (fCTC and CTC) showing a no difference in risk of death for CTC (p=0.95).

FIG. 6 is a set of images showing Cell fusion in PanIN and tumors from other organ sites. Solid tumors from women with previous sex-mismatched bone marrow transplantation permits analysis of cell fusion. Panel a shows Hematoxylin and Eosin stain of pancreatic ductal adenocarcinoma (PDAC) section. Panel b shows the same section with cytokeratin (gray), the Y-chromosome (Ychr, red) and Hoechst (blue) detection. The marked region in panel a is enlarged in panel c and contains pancreatic intraepithelial neoplasia (PanIN).Panels c-f show cytokeratin-positive cells with Y-chromosome-positive nuclei, white arrowhead. Representative areas boxed in white are enlarged. Bar=10 μm.

FIG. 7A is an image of karyotype and X- (red) and Y-chromosome (green) FISH analyses of parental macrophages (Mφ), unfused MC38 cancer cells and fusion hybrids. FIG. 7B is a diagram showing that fusion hybrids (gray sphere) cluster as a unique population based on their chromosome number and sex chromosomes, relative to McDs (white sphere) and MC38s (black sphere, arrows). FIG. 7C is a plot of karyotype analyses in Mφ, MC38 cells and 14d and 21d hybrids. Three hybrid isolates are shown. FIG. 7D shows the results of microarray analyses. Gray side bar marks hybrid gene expression that is similar to MC38 cancer cells, while black and white bars denote gene expression unique from MC38 cells. White bar marks hybrid gene expression that is similar to that in MDs. FIG. 7E is a chart of GO Analyses of differentially expressed genes in fusion hybrids versus MC38 cells that are similar to Mφ gene expression. Top 30 GO terms are displayed.

FIG. 8A is a graph and image showing cell confluence relative to time for MC38, Mφ, and hybrid cells. Still images from 48 and 96 h timepoints. FIG. 8B is a heatmap of relative adhesive preference for replicate MC38, Mφ and independent hybrid isolates determined by microenvironment microarray assay; hierarchical clustering according to relative preference for adhesion under 70 different microenvironmental conditions. FIG. 8C is a set of charts of mean cell confluence over time, and mean viability relative to untreated cells, for replicate MC38 and independent hybrid populations in the presence of increasing concentrations of Tgfβ2. FIG. 8D is a set of charts of mean cell confluence over time, and mean viability relative to untreated cells, for replicate MC38 and independent hybrid populations in the presence of increasing concentrations of Hgf. FIG. 8E is a chart of mean viability relative to untreated cells, for replicate MC38 and independent hybrid populations in the presence of increasing concentrations of Tgfβ1. FIG. 8F is a chart of mean viability relative to untreated cells, for replicate MC38 and independent hybrid populations in the presence of increasing concentrations of Tgfβ3. FIG. 8G is a chart of mean viability relative to untreated cells, for replicate MC38 and independent hybrid populations in the presence of increasing concentrations of TNF-α. FIG. 8H is an image showing the results of a scratch assay for relative migration of confluent MC38 and hybrid cultures and a graph showing quantification of migration over time. For FIGS. 8A-8H, *p<0.024.

FIG. 9A is a set of images showing B16F10 (H2B-RFP) cancer cells co-cultured with GFP-expressing McDs result in hybrid cells with RFP nuclei (red) and GFP-expressing cytoplasm (green). Unfused cancer cells only express RFP. Bar=10 μm. FIG. 9B is a set of two images showing lungs from mice injected with B16F10 cells and B16F10-derived hybrids. FIG. 9C is a graph of the results of chemotaxis assays towards Csf1 ligand. Hybrid chemotaxis towards Csf1 is statistically significant relative to unfused B16F10 cells and cells without ligand after 36h (*p<0.037). FIG. 9D is an image of gelatin zymography comparing Mmp2 activity in B16F10 cells and two different hybrid isolates.

FIG. 10A is a set of images showing a representative confocal micrograph of B16F10-derived fusion hybrid in a primary tumor visualized for YFP (yellow), RFP (red). FIG. 10B is a set of images showing isolated hybrid circulating tumor cells from B16F10 injected mouse blood visualized for YFP and RFP expression. FIG. 10C is a pie chart showing the results of FACS analyses of cell surface antigens on hybrid circulating tumor cells (GFP+/RFP+/CD45+; red scale). Wedge with asterisk denotes GFP+/RFP+/CD45− cells.

FIG. 11 is a diagram, images, and a flow cytometry plot showing that in vivo derived fusion hybrids can be isolated by FACS based on co expression of GFP and RFP. RFP expressing mammary tumor cells isolated from a transgenic PyMT mouse (Polyoma middle T) were injected into the mammary fat pad of an Actin-GFP transgenic mouse. Tumor cells isolated from a surgically removed 1 cm tumor were FACS-analyzed. Fusion hybrids (red box) expressed GFP and RFP. Unfused tumor cells only expressed RFP (black box), and represented the majority of tumor cells. Images of isolated cells are displayed in left panel.

FIG. 12 is a diagram and plot showing that FACS-isolated fusion hybrid tumor cells initiated tumor more readily than unfused tumor cells. To measure tumorigenecity, 2,500 FACS-isolated tumor cells, fused and unfused, were injected into the mammary fat pad of secondary recipient mice and monitored for growth for a period of 160 days. Fusion hybrids initiated tumor growth between 40-50 days and reached 1 cm in size by 50-80 days. Unfused tumor cells never initiated tumor growth.

### DETAILED DESCRIPTION

Terms:

Administration: To provide or give a subject an agent, such as a composition including a test compound or a macrophage-tumor cell fusion hybrid by any effective route. Exemplary routes of administration include, injection (such as subcutaneous, intramuscular, intradermal, intraperitoneal, and intravenous), oral, sublingual, rectal, transdermal, intranasal, vaginal and inhalation routes.

Antibody: A polypeptide including at least a light chain or heavy chain immunoglobulin variable region which specifically recognizes and binds an epitope of an antigen or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody. The VH and VL regions can be further segmented into complementarity determining regions (CDRs) and framework regions. The CDRs (also termed hypervariable regions) are the regions within the VH and VL responsible for antibody binding.

The term “antibody” encompasses intact immunoglobulins, as well the variants and portions thereof, such as Fab fragments, Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker. In dsFvs the chains have been mutated to introduce a disulfide bond to stabilize the association of the chains. The term also includes genetically engineered forms such as chimeric antibodies, heteroconjugate antibodies (such as, bispecific antibodies). See also, Pierce Catalog and Handbook, 1994-1995 (Pierce Chemical Co., Rockford, Ill.); Kuby, J., Immunology, 3rd Ed.,W.H. Freeman & Co., New York, 1997. The term also includes monoclonal antibodies (all antibody molecules have the same VH and VL sequences and therefore the same binding specificity) and polyclonal antisera (the antibodies vary in VH and VL sequence but all bind a particular antigen such as CD45.)

Binding: An association between two substances or molecules such as the association of an antibody with a polypeptide. As described herein, stable binding (or detectable binding) means that a macromolecule such as an antibody can bind to another macromolecule such as a polypeptide in a manner that can be detected. Binding can be detected by any procedure known to one skilled in the art, such as by physical or functional properties. Binding can also be detected by visualization of a label (such as a fluorescent label) conjugated to one of the molecules.

Specific binding means that a macromolecule such as an antibody binds to members of a class of macromolecules to the exclusion of macromolecules not in that class (binding to non-specific antibody binding macromolecules such as protein A, Fc receptors, etc. is excepted). A class of macromolecules can include macromolecules related by sequence or structure. For example, a pan-cytokeratin specific antibody can bind to some or all cytokeratins to the exclusion of other intermediate filament proteins. An antibody that binds specifically to a particular cytokeratin (such as cytokeratin 8) binds to that cytokeratin to the exclusion of other cytokeratins.

Biomarker (marker): Molecular, biological or physical attributes that characterize a physiological, cellular, or disease state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be any molecular structure produced by a cell or organism. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. Examples of biomarkers herein are lymphocyte/macrophage markers (proteins such as CD45 and CD68 that identify a cell as being of a lymphocyte or macrophage lineage) and circulating tumor cell markers (proteins such as CD49F, any of the members of the cytokeratin family, CD24, CD325, CD44, CD36, and that identify cells as being circulating tumor cells.) A biomarker can also be a discrete cellular entity such as a circulating tumor cell expressing particular cell surface markers including one or more of the markers above.

Cancer: A disease or condition in which abnormal cells divide without control and are able to invade other tissues. Cancer cells spread to other body parts through the blood and lymphatic systems. Cancer is a term for many diseases. There are more than 100 different types of cancer in humans. Most cancers are named after the organ in which they originate. For instance, a cancer that begins in the colon can be termed a colon cancer. However, the characteristics of a cancer, especially with regard to the sensitivity of the cancer to therapeutic compounds, are not limited to the organ in which the cancer originates. A cancer cell is any cell derived from any cancer, whether in vitro or in vivo.

Cancer is a malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc.

“Metastatic disease” or “metastasis” refers to cancer cells that have left the original tumor site and migrate to other parts of the body for example via the bloodstream or lymph system. The “pathology” of cancer includes all phenomena that compromise the well being of the subject. This includes, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

Circulating tumor cells (CTCs) are cells that are present in the circulation of patients with different solid malignancies. In some examples, they are derived from clones of the primary or metastatic tumor and are malignant. CTCs can be considered an independent diagnostic for cancer progression of carcinomas (Beitsch & Clifford, Am. J. Surg. 180, 446-449 (2000) (breast); Feezor et al, Ann. Oncol. Surg. 9, 944-53, 2002 (colorectal); Ghossein et al, Diagn. Mol. Pathol. 8, 165-175 (1999) (melanoma, prostate, thyroid); Glaves, Br. J. Cancer 48, 665-673, 1983 (lung); Matsunami et al, Ann. Surg. Oncol. 10, 171-175, 2003 (gastric); all of which are incorporated by reference herein).

Detection and enumeration of circulating tumor cells is important for patient care for a number of reasons. They may be detectable before the primary tumor, thus allowing early detection of disease stage diagnosis. The number of CTC's in circulation have been shown to decrease in response to therapy, so the ability to enumerate CTCs allow one to monitor the effectiveness of a give therapeutic regimen. They can be used as a tool to monitor for recurrence in patients with no measurable disease in the adjuvant setting. As disclosed herein, circulating tumor cells include cells that express one or more macrophage markers and one or more tumor cell markers.

Contacting: Placing within an environment where direct physical association occurs, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Cytokeratins: Structural proteins that belong to the intermediate filament (IF) family of proteins with a number of uses in epithelial cells. As of filing of this disclosure, 23 types of cytokeratin are known with different cytokeratins having been shown to be markers of particular types of cancer and/or cancer activities including cytokeratin 5, cytokeratin 7, cytokeratin 8, cytokeratin 10, cytokeratin 13, cytokeratin 17, and cytokeratin 18 (Moll et al; Cell 31: 11-24 (1982); Varadhachary et al, Cancer 100, 1776-1785 (2004). Gusterson et al, Breast Cancer Res 7, 143-148 (2005); Kanaji et al, Lung Cancer 55, 295-302 (2007). Moll et al, Virchows Arch B Cell Pathol Incl Mol Pathol, 58, 129-145 (1989); Rugg et al, J Invest Dermatol , 127: 574-580 (2007); Betz et al, Am J Human Genet 78, 510-519 (2006); Ramaekers et al, Am J Pathol 136, 641-655 (1990); Yabushita et al, Liver 21, 50-55 (2001); Chatzipantelis et al, Hepatol Res 36, 182-187 (2006); Galarneau et al, Exp Cell Res 313, 179-194 (2007); Ku & Omary, J Cell Biol 174, 115-125 (2006); Lau & Chiu, Cancer Res 67, 2107-2113 (2007); Linder et al, Cancer Lett 214, 1-9 (2004); van Dorst et al, J Clin Pathol 51, 679-684 (1988); Maddox et al, J Clin Pathol 52, 41-46 (1999); Toyoshima et al, J Cancer Res Clin Oncol, 134: 515-521 (2008); Deshpande et al, Am J Surg Pathol 28, 1145-1153 (2004); Park et al, J Korean Med Sci 22, 621-628 (2007); Barroeta et al, Endocr Pathol 17, 225-234 (2006); Ignatiadis et al, J Clin Oncol 25, 5194-5202 (2007). Lindberg & Rheinwald, Am J Pathol 134, 89-98 (1989); www.antibodies-online.com/resources/18/624/cytokeratins-in-the-detection-of-tumors/ last accessed 10 Oct. 2016) all of which are incorporated by reference herein). Cytokeratins can be detected through the use of specific antibodies such as an antibody to a specific human or mouse cytokeratin or through the use of a pan-cytokeratin antibody that detects more than one, more than three, more than 5, more than 10, more than 12, more than 15, or more than 20 cytokeratin molecules using a single antibody.

Fluorescent protein: A protein characterized by a barrel structure that allows the protein to absorb light and emit it at a particular wavelength. Fluorescent proteins include green fluorescent protein (GFP) modified GFPs and GFP derivatives and other fluorescent proteins, such as EGFP, EBFP, YFP, RFP, BFP, CFP, ECFP, mCherry, and circularly permutated fluorescent proteins such as cpVenus.

Label: A label can be any substance capable of aiding a machine, detector, sensor, device, column, or enhanced or unenhanced human eye from differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include: radioactive isotopes or chelates thereof; dyes (fluorescent or nonfluorescent), stains, enzymes, nonradioactive metals, magnets, protein tags, fluorescent proteins, any antibody epitope, any specific example of any of these; any combination between any of these, or any label now known or yet to be disclosed. A label may be covalently attached to a biomolecule or bound through hydrogen bonding, Van Der Waals or other forces. A label may be covalently or otherwise bound to the N-terminus, the C-terminus or any amino acid of a polypeptide or the 5′ end, the 3′ end or any nucleic acid residue in the case of a polynucleotide.

One particular example of a label is a small molecule fluorescent dye. Such a label can be conjugated to an antibody such as an antibody that binds a macrophage or tumor cell marker. One of skill in the art would be able to identify and select any appropriate fluorescent dye or combination of fluorescent dyes for use in the disclosed methods.

Another particular example of a label is a protein tag. A protein tag includes a sequence of one or more amino acids that may be used as a label as discussed above, particularly for use in protein purification. In some examples, the protein tag is covalently bound to the polypeptide. It may be covalently bound to the N-terminal amino acid of a polypeptide, the C-terminal amino acid of a polypeptide or any other amino acid of the polypeptide. Often, the protein tag is encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG®, V5, c-Myc, HA-tag, and so forth.

A His-tag facilitates purification and binding to on metal matrices, including nickel matrices, including nickel matrices bound to solid substrates such as agarose plates or beads, glass plates or beads, or polystyrene or other plastic plates or beads. Other protein tags include BCCP, calmodulin, Nus, Thioredoxin, Streptavidin, SBP, and Ty, or any other combination of one or more amino acids that can work as a label described above.

Another particular example of a label is biotin. Biotin is a natural compound that tightly binds proteins such as avidin or streptavidin. A compound labeled with biotin is said to be ‘biotinylated’. Biotinylated compounds can be detected with avidin or streptavidin when that avidin or streptavidin is conjugated another label such as a fluorescent, enzymatic, radioactive or other label.

Macrophage: A macrophage (interchangeably abbreviated herein as Mφ) is a phagocytic, mononuclear, myeloid, cell of the immune system. Macrophages can be found in and purified from the peripheral blood, spleen, and lymph nodes. Alternatively, human and mouse macrophage cell lines are available. Macrophages can be identified by cell surface markers including CD14, CD33, CD45, CD11b/Mac-1, Ly-71 (F4/80), Csf1R, CD68, and others known in the art.

Macrophage—tumor cell fusion hybrid: Herein, these are interchangeably termed, circulating tumor cells, fused CTC's, fusion derived CTC's, MφP-cancer cell fusion hybrids, Mφ-cancer cell fusions, fusion hybrids, cell fusion hybrids, MφP-tumor cell fusion hybrids, (tumor cell line)-derived hybrids, hybrids, including any similar terms used in the Examples below.

Macrophage-tumor cell fusion hybrids are fusions between a tumor cell and a macrophage that occurs when a macrophage and a tumor come in contact with one another. Macrophage-tumor cell fusion hybrids can be isolated from human or animal subjects as described in detail herein. Alternatively, macrophage-tumor cell fusion hybrids can be constructed in vitro by contacting primary macrophages or a macrophage cell line with a primary tumor or tumor cell line. Cellular fusions between a macrophage and a tumor cell artificially produced in vitro using a reagent that promotes membrane fusion (such as fusogenic or syncytium forming viruses or polypeptides derived from those viruses) are not considered to be macrophage-tumor cell fusion hybrids as described herein.

Without being bound by theory, fusion with macrophages and potentially other myeloid or lymphoid cells provides a mechanism for how tumor cells can rapidly gain cellular behaviors attributed to aggressive macrophage-like activities that facilitate metastatic spread of cancer. Such activities include extracellular matrix remodeling, survival in circulation, seeding of distant metastatic sites, and the development of cellular heterogeneity that contributes to disease progression and/or treatment resistance.

Macrophage-tumor cell fusion hybrids can be identified by the expression of one or more macrophage markers such as those described above and one or more tumor cell markers such as such as one or more of the cytokeratins, CD49F, CD24, CD325, CD44, CD11b, CSF1 R, or CD36 as well as others known in the art or yet to be disclosed.

Circulating cells that express both macrophage and tumor cell markers in humans are hypothesized to be macrophage-tumor cell fusion hybrids, and can be described as such herein, but data that prove that such cells result from a mechanism of macrophage-tumor cell fusion hybrid using mouse models as described herein are unavailable.

Polypeptide: Any chain of amino acids, regardless of length or posttranslational modification (such as glycosylation, methylation, ubiquitination, phosphorylation, or the like). “Polypeptide” is used interchangeably with peptide or protein, and is used to refer to a polymer of amino acid residues. A “residue” refers to an amino acid or amino acid mimetic incorporated in a polypeptide by an amide bond or amide bond mimetic.

Purification: Purification of a cell can be achieved by any method known in the art including by use of methods that involve the use of labeled antibodies that bind cell surface antigens such as fluorescence activated cell sorting, sorting through the use of magnetic beads, or on purification columns. Purification does not require absolute purity (that is the purified cells are exactly 100% cells of the desired type.) Instead, a purified population of cells can include at least 60%, 70%, 80%, 90%, 95%, 98%, 99% 99.9%, or 99.99% cells of the desired type.

Subject: A living multicellular vertebrate organism, a category that includes, for example, mammals and birds. A “mammal” includes both human and non-human mammals, such as mice. In some examples, a subject is a patient, such as a patient diagnosed with cancer. In other examples, a subject is a patient yet to be diagnosed with cancer.

Test compound: Any small molecule, natural product, natural or artificial polypeptide, natural or artificial polynucleotide (including an oligonucleotide), aptamer, RNAi based molecule (siRNA, shRNA, miRNA or any mutant or mimic thereof), or any other molecule that has or can conceivably have an effect upon a cell or tissue in vitro or on a cell, tissue, organ, or organism in vivo.

The ability of a test compound to have a desired effect on a cell, tissue, organ, or organism is assessed in a screen. For example, test compounds can be screened for their ability to inhibit one or more activities of macrophage tumor-cell fusions. Such screens can be performed in acellular systems such as tests of phosphorylation of a particular target, in cellular systems, or in living animals.

A test compound is generally provided in a vehicle, such as a solvent. The vehicle can be any appropriate solvent including compositions including water, ions, or organic compounds. Examples of vehicles include buffered saline or other buffered solvents or DMSO or other organic solvents.

The methods herein can be used to screen a plurality of test compounds, also described as a library of test compounds. Methods disclosed herein can be further adapted to high throughput screening of a set of test compounds in batches of 96, 384, 1048, or more on assay plates adapted for such screening.

Test compounds also encompass negative controls such as vehicle only controls or controls including a compound similar to the test compound but known not to have the desired effect (such as a structurally similar antibody with specificity for another macromolecule or an irrelevant siRNA) as well as positive controls which are compounds already known to have the desired effect on the cell, tissue or organism.

Tumor: All neoplastic cell growth and proliferation, whether malignant or benign, and all pre-cancerous and cancerous cells and tissues. Tumor markers include polynucleotides and polypeptides expressed by tumors to a greater extent than they are expressed by non-tumor cells, including cell surface tumor antigens.

Methods of identifying and isolating/purifying circulating tumor cells. Disclosed are methods of identifying circulating cells that express both macrophage and tumor markers in a subject (including macrophage-tumor cell fusion hybrids). Such methods involve contacting a cell taken from the blood of the subject with an antibody that specifically binds a macrophage marker. The method further involves contacting the cell with an antibody that specifically binds a tumor antigen. Both antibodies are labeled and the labels are distinguishable from one another. A cell with detectable expression of both the first label and the second label is identified as such. The antibody that binds to the macrophage marker can bind to any macrophage marker either described herein or known in the art. In but one example, the antibody that binds to the macrophage marker binds CD45. Similarly, the antibody that binds to the tumor antigen can bind to any tumor antigen either described herein or known in the art. In but one example, the antibody that binds to the tumor antigen binds to a cytokeratin. In a more specific example, the antibody that binds to the tumor antigen is a pan-cytokeratin antibody.

The labels can be selected from any label described herein or otherwise known in the art. In some examples the labels are fluorescent labels that are distinguishable from one another on the basis that they emit light at different wavelengths. Such fluorescent labels allow purification of macrophage tumor cell fusion hybrids by any of a number of methods including fluorescence activated cell sorting.

In another example, one of the labels, such as the label conjugated to the antibody that binds to the macrophage marker, is a non-fluorescent label such as a magnetic bead. The cells expressing both macrophage and tumor cell markers are purified (along with all other macrophages) by retaining the magnetic bead labeled cells using a magnetic field. The resulting cells are then contacted with the labeled antibody that binds to the tumor antigen and purified by any of a number of methods including fluorescence activated cell sorting, purification in a magnetic field or another method known in the art. One of skill in the art will be able to apply other known methods of isolating antibody labeled cells to the methods disclosed herein without undue experimentation.

Methods of screening test compounds. One method of screening a test compound for inhibition of activities of an in vitro derived macrophage-tumor cell fusion hybrid involves contacting an in vitro derived macrophage-tumor cell fusion hybrid with the test compound, contacting another in vitro derived macrophage-tumor cell fusion hybrid with a negative control and measuring one or more activities of the macrophage-tumor cell fusion hybrid that was contacted with the test compound and of the macrophage-tumor cell fusion hybrid that was contacted with the negative control. Activities include cellular proliferation, cellular migration, metastasis, chromosomal loss, extracellular matrix remodeling, and/or survival in circulation. If the macrophage-tumor cell fusion hybrid administered the test compound displays less activity than the macrophage-tumor cell fusion hybrid administered the negative control (e.g. less cellular proliferation, migration, metastasis, chromosomal loss ECM remodeling, and/or survival in circulation, then that is an indication that the test compound inhibits the one or more activities of the in vitro derived macrophage-tumor cell fusion hybrid. Conversely, and as understood by one of ordinary skill in the art, positive controls can also be used.

In vitro derived macrophage-tumor cell fusion hybrids can be generated in vitro by contacting a macrophage with a tumor cell in culture, provided that the culture lacks an agent that promotes cell-cell fusion such as a virus that forms syncytia and/or any fusogenic peptides (whether artificial or derived from syncytia forming viruses.) In some examples, the macrophage can express a first marker protein and the tumor cell can express a second marker protein. In these examples, macrophage-tumor cell fusion hybrids are identified based on the expression of the marker proteins, provided that the marker proteins are distinguishable from one another.

A marker protein can be any protein expressed by the cell including an endogenously expressed protein or an exogenous protein (not native to the species from which the cell was derived) that the cell was engineered to express by recombinant DNA technology. In some examples, the marker proteins are exogenous fluorescent proteins such as RFP, GFP, YFP, CFP, mCherry, or any other GFP derived proteins expressed in the cells, provided that the macrophage and the tumor cell do not both express the same fluorescent protein. The macrophage and the tumor cell can be made to express an exogenous marker protein through any of a number of methods known in the art. For example, the cells can be transiently or stably transfected with an expression vector including a promoter operably linked to a nucleic acid that encodes the marker protein. The promoter can be a constitutively active promoter or a cell specific promoter (e.g. a promoter that causes expression of the protein only in macrophages.) Such an expression vector also contains other nucleic acid sequences that aid in cloning, selection of positive clones, and control of expression. Transfection of such an expression vector can be performed by electroporation, delivery through viral infection, by use of transfection reagents such as lipid based transfection agents, or by any method known in the art.

Activities of in vitro derived macrophage-tumor cell fusion hybrids can be assessed by any of a number of methods including an MTS assay, a chemotaxis assay, a scratch wound assay, injection of the macrophage-tumor cell fusion hybrids into the spleen of an experimental animal and measuring liver metastases, any methodology disclosed in the Examples below, or any appropriate methodology disclosed in the art.

The macrophage and/or tumor cells can be derived from any subject including a human or an experimental animal such as a mouse, rat, rabbit, guinea pig, or non-human primate. In some examples, the macrophage is derived from a transgenic animal (such as a mouse) engineered to express a fluorescent protein in its macrophages. Expression can be limited to macrophages, limited to other cells of the myeloid lineage, limited to hematopoietic cells, or expressed in all cells, including macrophages. The tumor cell can be derived from any type of tumor from any species. Examples of types of tumors from which cells can be derived include Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sézary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sézary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenström macroglobulinemia and Wilms tumor (kidney cancer).

An indication that the test compound inhibits one or more activities of an in vitro derived macrophage-tumor cell fusion hybrid is not a guarantee that the test compound will inhibit macrophage-tumor cell fusion hybrids in vivo, much less serve as a clinical compound. Rather, an indication is a signal to one of skill in the art to pursue further investigation of the test compound and can result in further activities such as validation of the test compound in another model (such as an in vivo model) and the generation of compounds related to the test compounds (such as mutants or small molecule derivatives).

Other methods of screening compounds are disclosed. Such methods include screening test compounds for inhibition of proliferation or metastasis of in vivo derived macrophage-melanoma cell fusion hybrids or macrophage-mammary tumor cell fusion hybrids. The method involves injecting a melanoma cell line or primary mammary tumor into a mouse. The melanoma line or primary mammary tumor is engineered to express a fluorescent protein. The mouse has been engineered to transgenically express a fluorescent protein that is distinguishable from the fluorescent protein expressed by the melanoma line or primary mammary tumor. The melanoma line or primary mammary tumor cells expand to form a tumor in the mouse. The tumor is removed from the mouse. Cells expressing both the first fluorescent protein and the second fluorescent protein are purified from the tumor by an appropriate method (such as flow cytometry.) The cells expressing the first fluorescent protein and the second fluorescent protein are then injected into a group of mice that have been or will be treated with the test compound (including simultaneous injection of the cells and administration of the test compound.) The cells expressing both the first fluorescent protein and the second fluorescent protein are injected into another group of mice that have been and/or will be left untreated or have been or will be treated with a negative control. If proliferation and/or metastasis occurs to a lesser degree in the mouse administered the test compound, then that is an indication that the test compound inhibits proliferation or metastasis in an in vivo derived macrophage-melanoma cell fusion hybrid.

The mice administered the cells that express both the first fluorescent protein and the second fluorescent protein can be injected with an appropriate number of cells. The number can be at least 50 cells, at least 100 cell fusions at least 200 cell fusions, at least 500 cell fusions, at least 1000 cell fusions, at least 2000 cell fusions, at least 3000 cell fusions, at least 5000 cell fusions, or at least 10,000 cell fusions or any range from 50-10,000 cell fusions. The tumor can be removed at an appropriate size range including from 0.25-5 cm in diameter, 0.5-3 cm in diameter, from 1-2 cm in diameter, or any narrower range.

In some examples, the melanoma line used is a mouse melanoma line, such as a B16F10 line, or primary mouse melanoma. In other examples, the primary mammary tumor is derived from a transgenic PyMT mouse.

In other examples of the method, the melanoma cells used are primary human melanoma cells, a human melanoma cell line, a human breast cancer cell line, or primary human breast cancer cells, provided that the transgenic mice into which the tumors are first introduced and the recipient mice into which the cells that express the first fluorescent protein and the second fluorescent protein are introduced are immunocompromised. Examples of immunocompromised mice include Rag1 knockouts, Rag2 knockouts, scid mice, athymic nude mice, and others known in the art. In still further examples, the mice into which the primary human tumor or human cell lines are mice with a humanized immune system—mice that lack a fully mouse immune system but have been engrafted with some or all components of a human immune system. These include CD34+humanized mice and PBMC humanized mice.

The test compound can be administered at any time relative to the macrophage-melanoma cell fusion hybrids. It can be administered prior to, concurrently with, or after the macrophage-melanoma cell fusion hybrids. The test compound can also be administered by any appropriate route. One of skill in the art in light of this disclosure can select the proper route to administer a test compound and/or a macrophage-tumor cell fusion hybrid without undue experimentation.

Methods of assessing the chance of survival of a human subject with pancreatic cancer. Disclosed are methods of assessing the survival of a human subject with pancreatic cancer, particularly the one year survival of a patient presenting with pancreatic cancer. The method involves receiving a peripheral blood sample from the subject. The sample includes intact mononuclear cells. The method further involves contacting the blood sample with an anti-CD45 antibody. The antibody further includes a first fluorescent label. The blood sample is further contacted with an anti-pan-cytokeratin antibody. The anti-pan-cytokeratin antibody further includes a second fluorescent label distinguishable from the first fluorescent label. The blood sample is also contacted with Hoescht stain. The number of CD45+/cytokeratin+/Hoescht+ cells is counted in at least a subset of the cells in the sample and the percentage of CD45+/cytokeratin+/Hoescht+ cells is calculated. If more than 0.8% of the cells counted are CD45+/cytokeratin+/Hoescht+ cells, then that is an indication that the subject has a less than 12% chance of survival past one year.

The number of cells counted need not be all the cells in the sample. A subset of cells can be counted. One of skill in the art in light of this disclosure can calculate an appropriate number of cells to count given the above percentages to achieve a statistically appropriate sample size. Appropriate numbers of cells to count in the subset include at least 500, at least 1000, at least 2000, at least 3000, at least 5000, or any number less than the entire number of cells in the sample.

The method can further involve adhering the blood sample to a glass substrate (such as a microscope slide) and fixing the cells in 4% paraformaldehyde prior to contacting the blood sample with one or both antibodies.

The blood sample can also be any fraction of blood including mononuclear cells. One such fraction is the so-called “buffy coat” fraction made by passing whole blood over a Ficoll 0 gradient.

### EXAMPLES

The following examples are for illustration only. In light of this disclosure, those of skill in the art will recognize that variations of these examples and other examples of the disclosed invention be possible without undue experimentation.

### Example 1

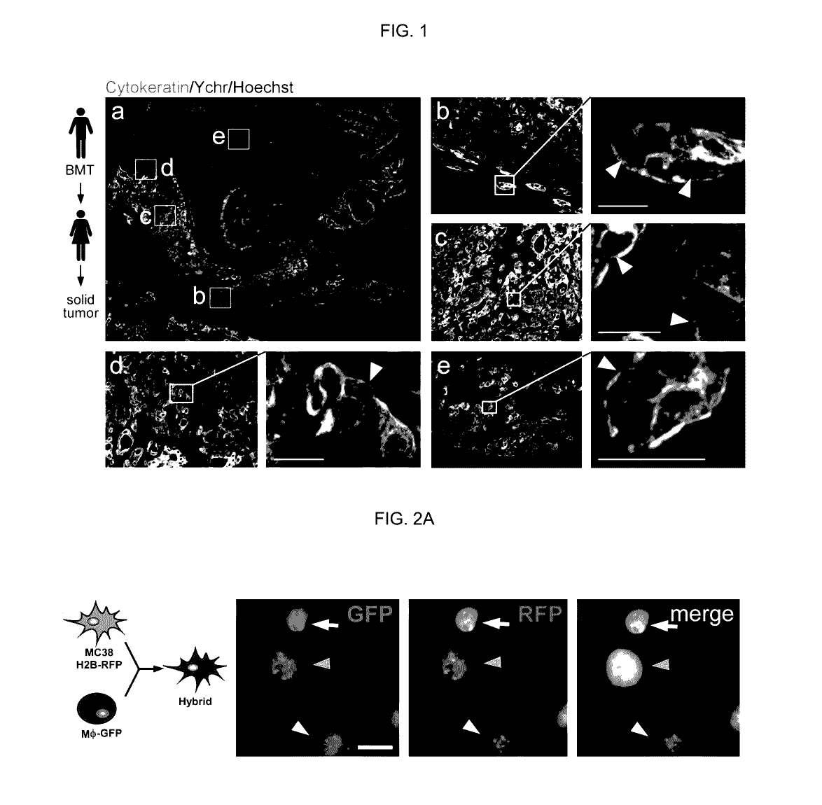

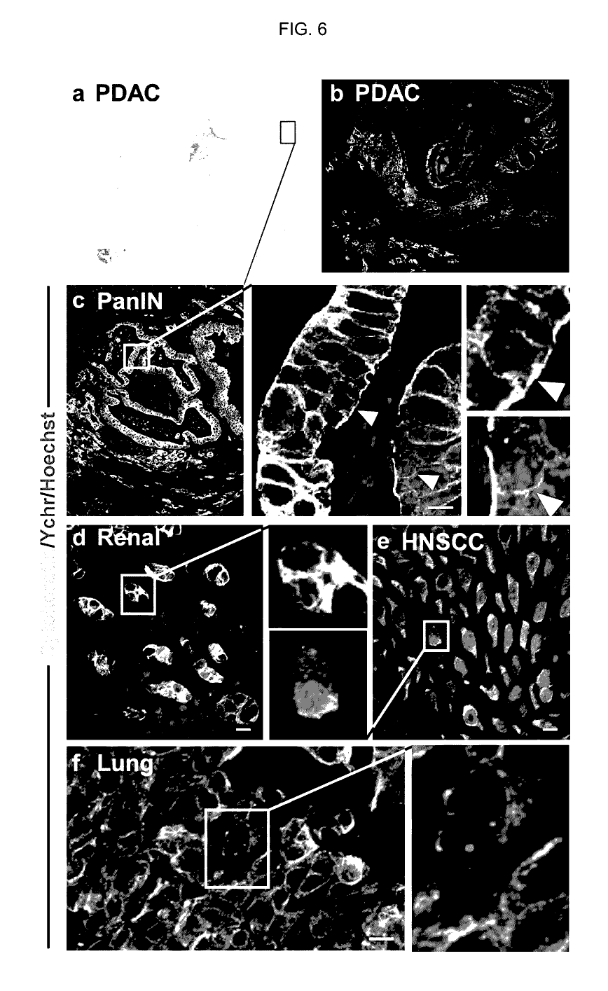

Cell Fusion in Human Tumors. To establish that cell fusion occurs in human cancers, a system that identifies a blood cell marker in the context of the tumor epithelium (Silk et al, PLoS One 8, e55572 (2013); incorporated by reference herein) was used. Specifically, tumor biopsies from female patients who previously underwent a sex-mismatched bone marrow transplant (BMT) but subsequently developed a secondary solid tumor were analyzed. Tumor epithelia were identified with pan cytokeratin antibodies and interrogated with fluorescence in-situ hybridization (FISH) probes to the Y-chromosome to reveal fusion between the tumor cell and the transplanted male hematopoietic cell (FIGS. 1, 6). In a patient biopsy of pancreatic ductal adenocarcinoma (PDAC), cancer cells that contained nuclei harboring a Y-chromosome were readily detectible in regions of pancreatic cancer (FIGS. 1A-1E, 6A, 6B), as well as in pre-neoplastic areas of pancreatic intraepithelial neoplasia (PanIN). Confocal analyses of these cells confirmed that the Y-chromosome was located in the nucleus of a cytokeratin-positive tumor cell. These Y-chromosome-positive tumor epithelial cells are not unique to PDAC, as they were also detected in various other solid tumors, including renal cell carcinoma, head and neck squamous cell carcinoma, and lung adenocarcinoma (FIGS. 6D-6F). These observations are consistent with previous reports of cell fusion in human cancer that used various detection methods (Lorico 2015 supra and Lazova et al, Adv Exp Med Biol 714, 151-172 (2011); incorporated by reference herein), as well as a report of Mφ-cancer cell fusion in a mouse model of intestinal cancer (Powell 2011 supra).

### Example 2

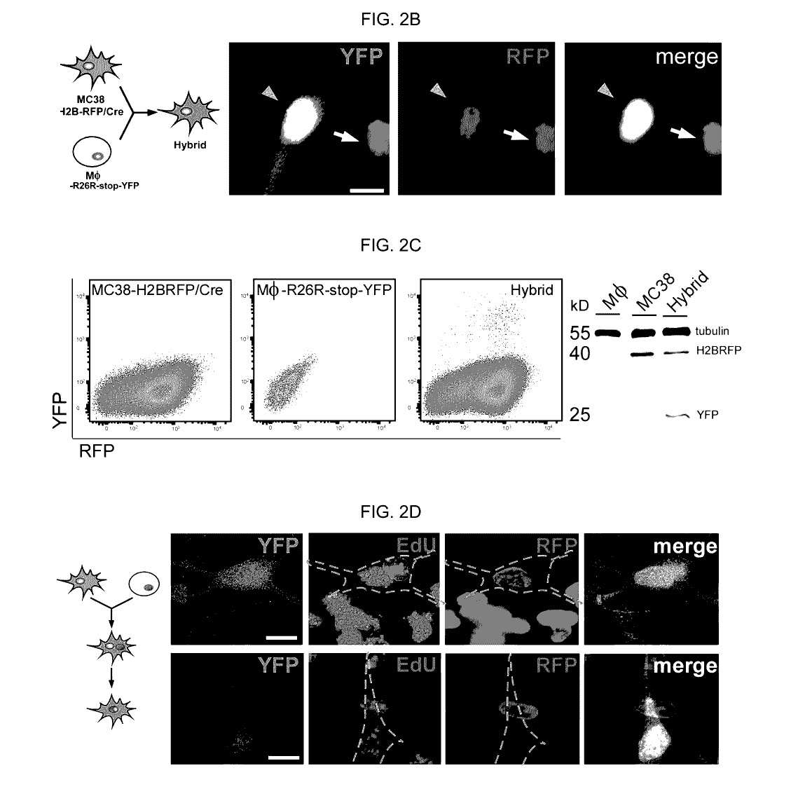

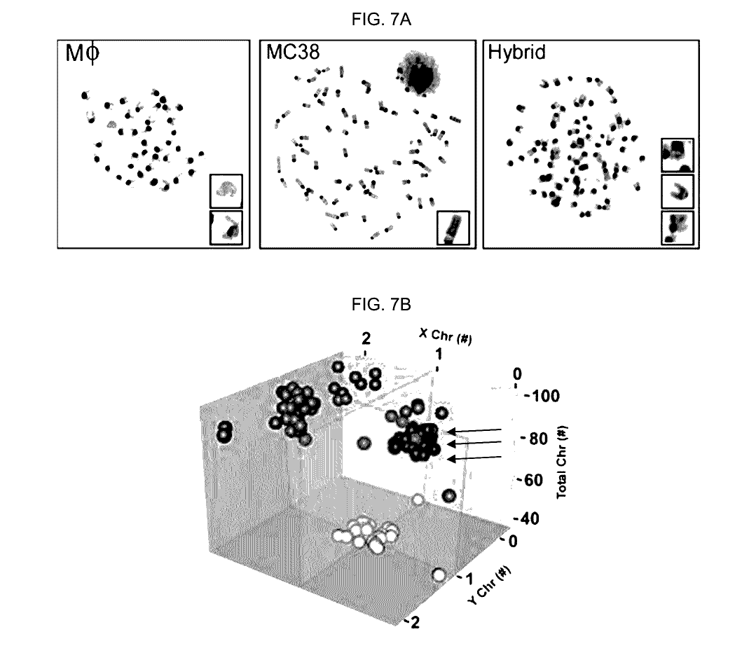

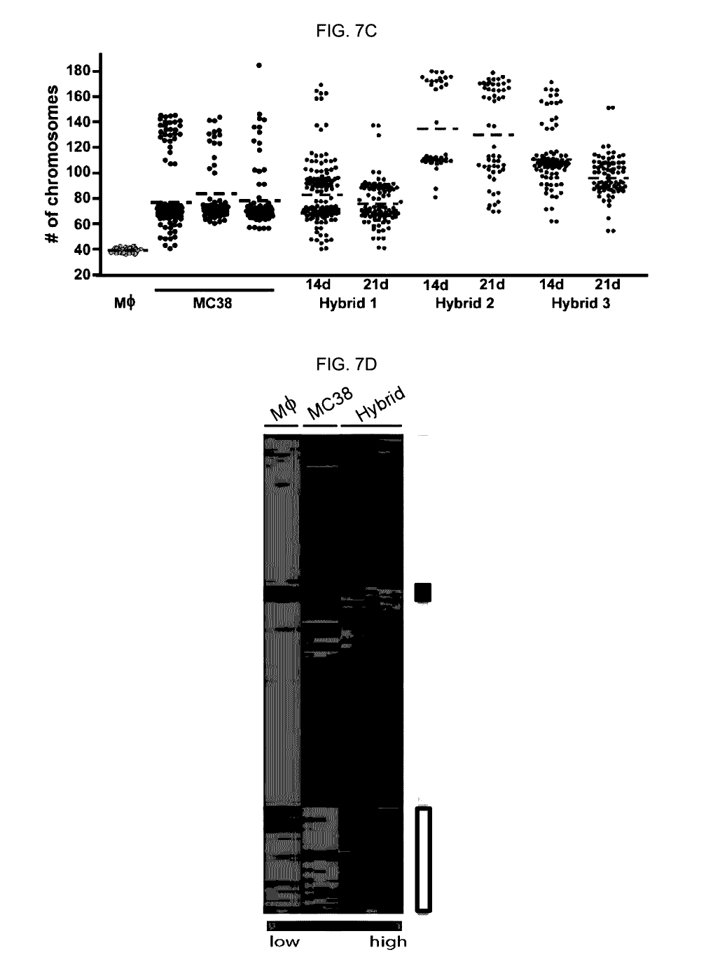

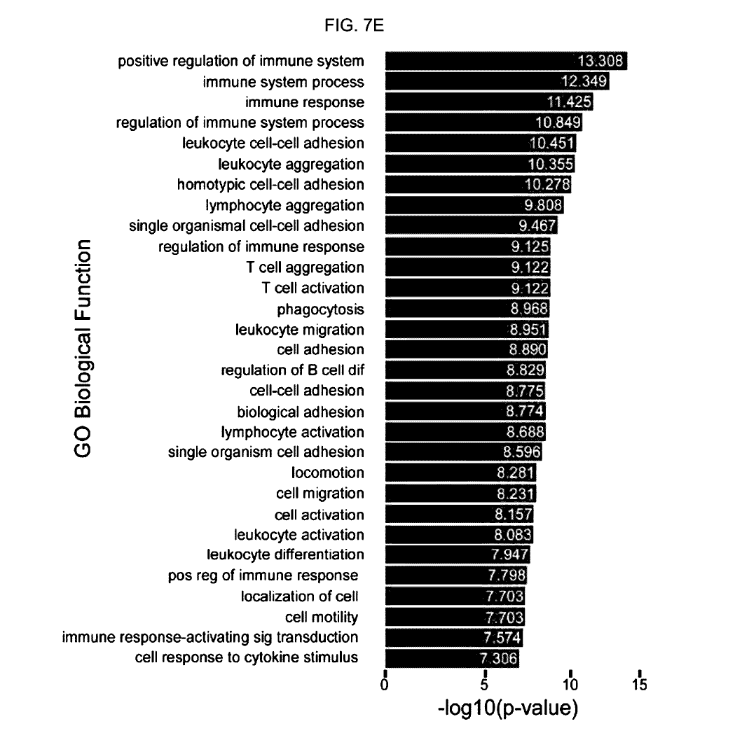

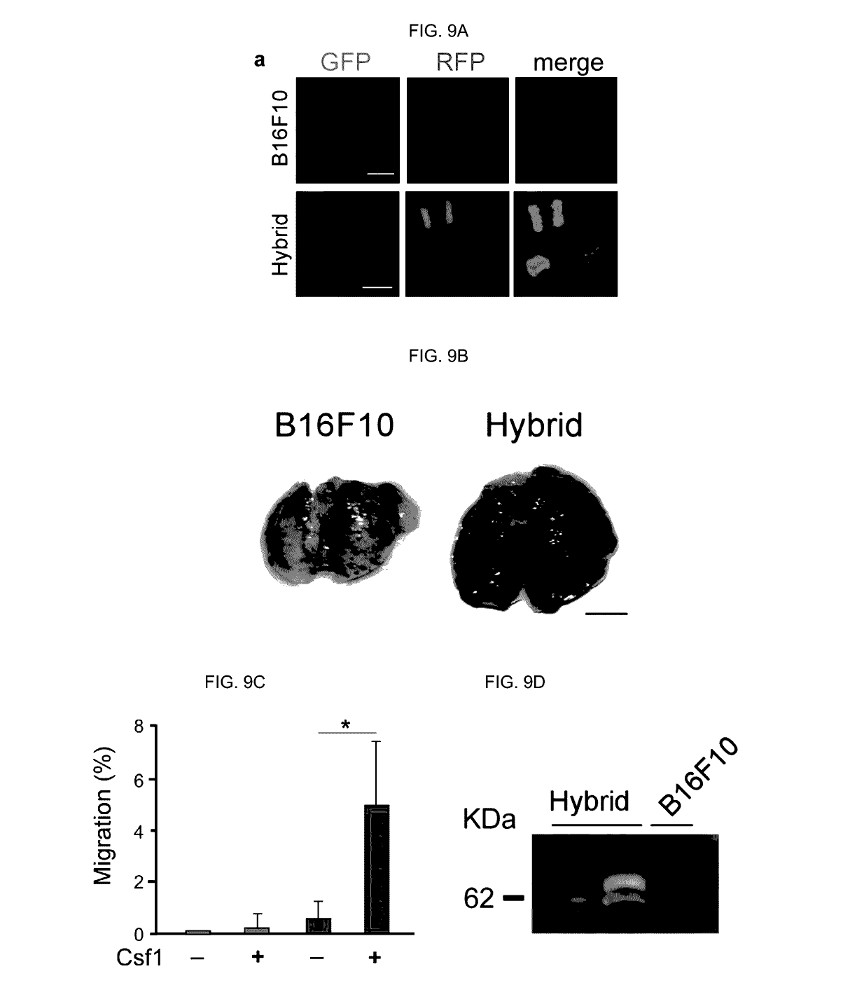

In vitro-derived macrophage tumor cell fusion hybrids display bi-parental lineage. Based upon the previous discovery that Mφs are the most prominent immune cell fusion partner for epithelial cells (Powell 2011 supra), in vitro validation and analyses of Mφ-cancer cell fusion hybrids were performed. Two mouse cancer cell lines (colon adenocarcinoma, MC38, and melanoma, B16F10) were engineered to stably express a Cre recombinase and histone 2B fused to red fluorescent protein (H2B-RFP). In co-cultures, these cancer cells spontaneously fused with bone marrow-derived MOs isolated from transgenic mice expressing Actin-GFP (Okabe et al, FEBS Lett 407, 313-319 (1997); incorporated by reference herein) or the YFP Cre reporter (Srinivas et al, BMC Dev Biol 1, 4 (2001); incorporated by reference herein) to produce MO-cancer fusion hybrids. Fusion hybrids were identified by their co-expression of nuclear RFP and cytoplasmic GFP, or YFP (FIGS. 2A, 2B, 9A). Hybrid cells could be FACS-isolated with high purity based upon YFP expression (FIG. 2C). To demonstrate the biparental lineage of these hybrid cells, three different approaches were used. First, Mφs labeled with 5-ethynyl-2′-deoxyuridine (EdU) prior to co-culture with cancer cells produced Mφ-cancer cell fusion hybrids that initially harbored two nuclei, one labelled with EdU, of Mφ origin, and the other expressing H2B-RFP, of cancer cell origin (FIG. 2D). Importantly, upon first mitotic division, bi-nucleated hybrids underwent nuclear fusion and contained a single nucleus with both EdU-labelled and H2B-RFP-labelled DNA (FIG. 2D). A second approach, using karyotype analysis of sex-chromosomes in male-isolated MOs (XY) fused to cancer cells (XO), revealed that fusion hybrids contained three sex chromosomes (XXY; FIG. 7A), consistent with fusion between the Mφ and cancer cell.

Chromosome counting demonstrated that fusion hybrids clustered as a unique cell population defined by total chromosome number and sex-chromosome content (FIG. 7B). Interestingly, loss of chromosomes was observed in many of the fusion hybrids (FIGS. 7B, 7C), suggesting fusion as a mechanism of rapidly amplifying tumor heterogeneity. Consistent with this observation, fusion hybrids analyzed after the fusion event contained chromosome numbers representing complements of both parent cells, but with continued passaging, the hybrids lost chromosomes before settling in a hyperdiploid state (FIG. 7C). Finally, transcriptome analysis of Mφ-cancer cell fusion hybrids revealed that these cells predominantly displayed cancer cell transcriptional identity, but also notably retained a Mφ gene expression signature (FIG. 7D). Interestingly, 5,827 genes were differentially expressed in MC38 cancer cells relative to both Mφ and cell fusion hybrids (FIG. 7D) and were clustered into GO Biologic functions that were attributed to macrophage behavior (FIG. 7E). Together, these findings support the notion that cell fusion between MφDs and cancer cells produces a distinct hybrid cell type that shares characteristics of both parental derivatives.

### Example 3

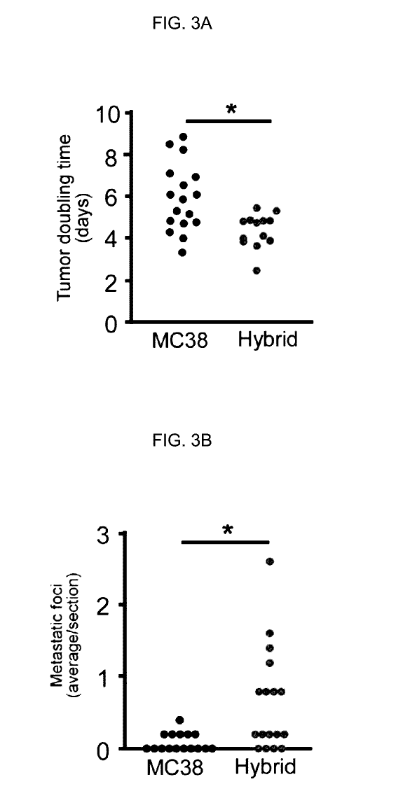

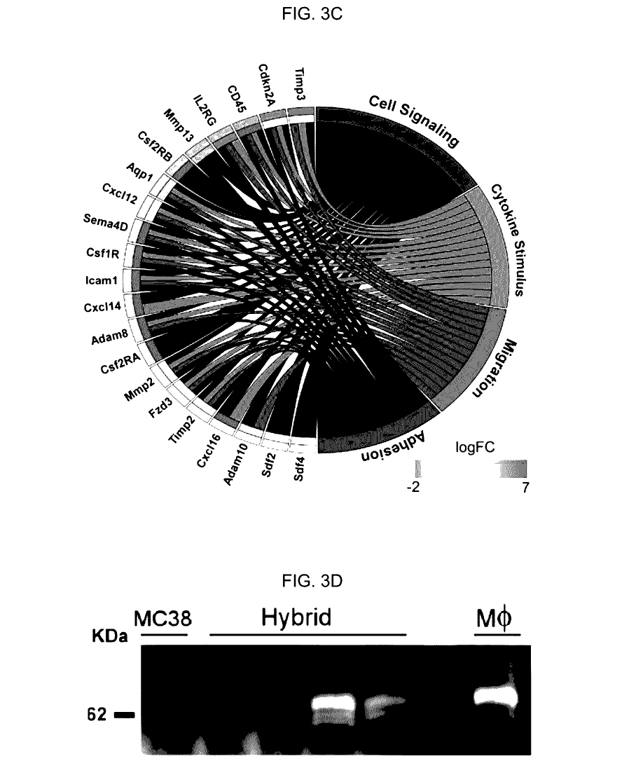

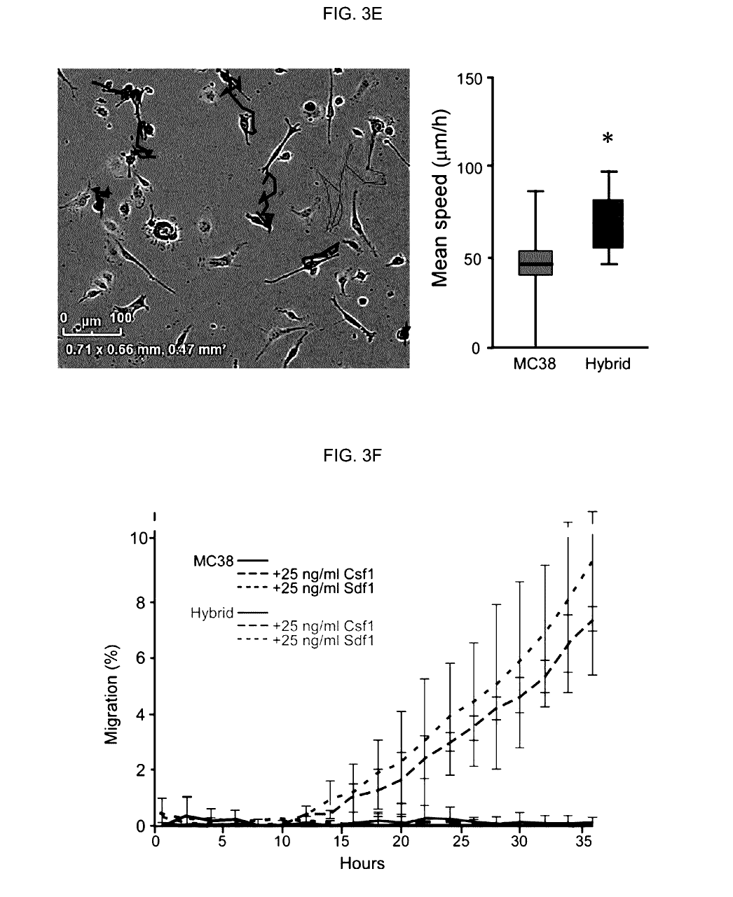

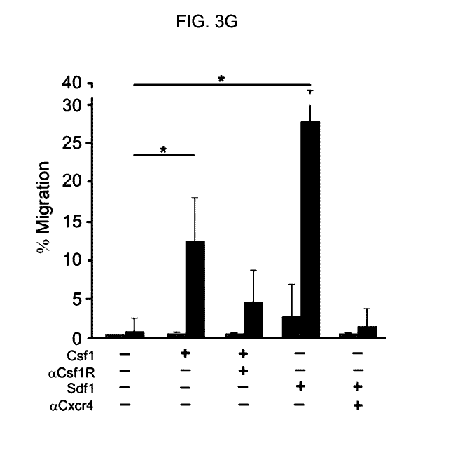

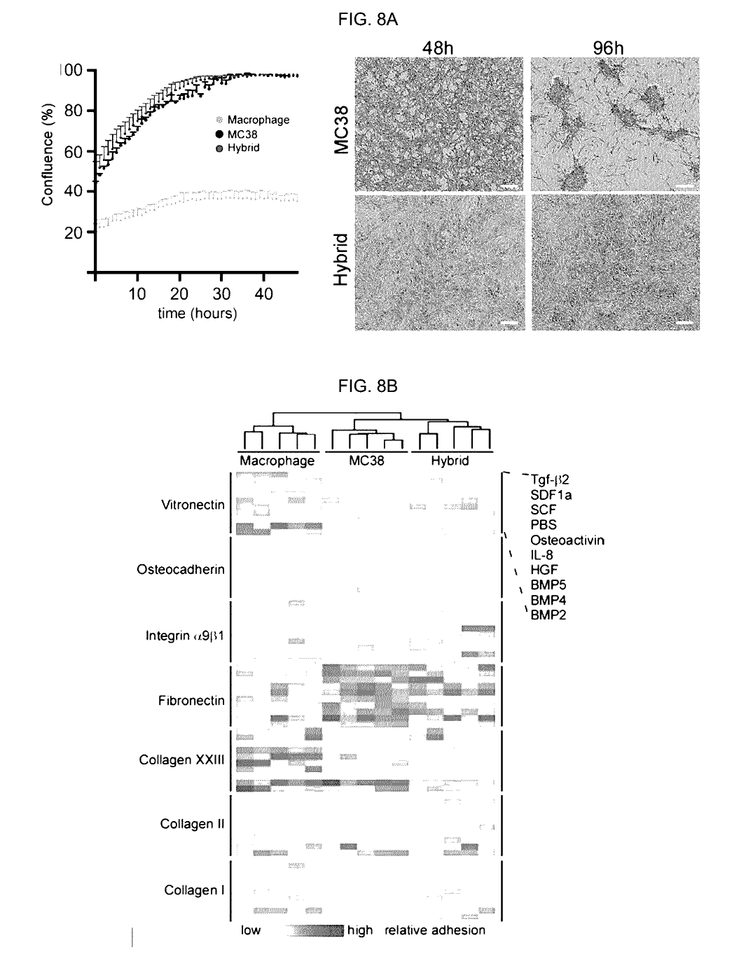

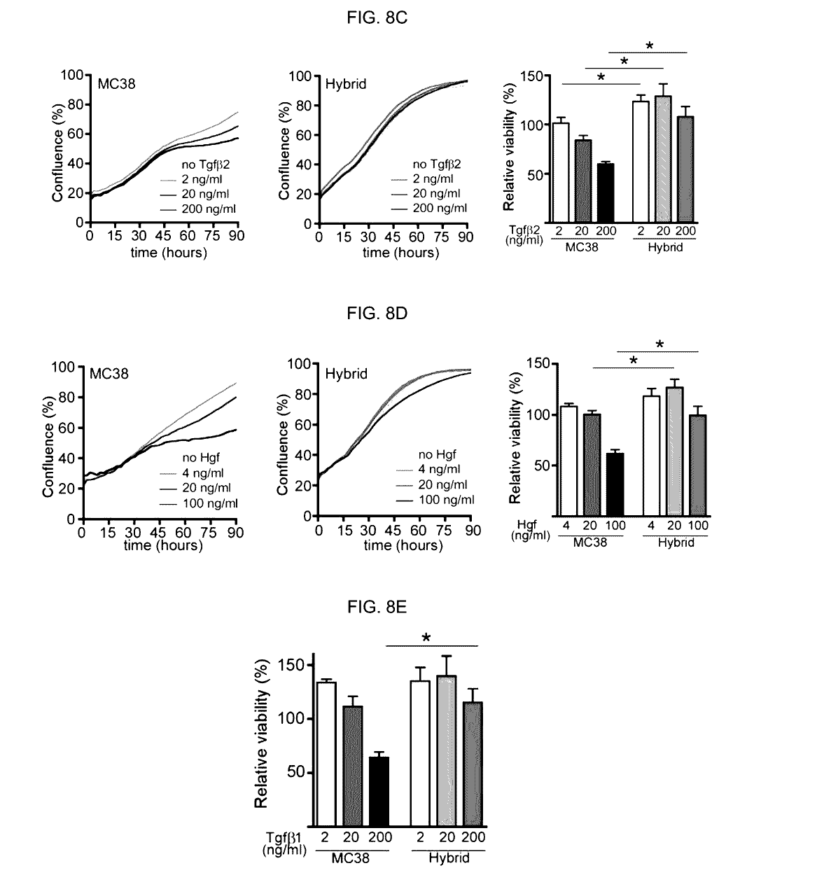

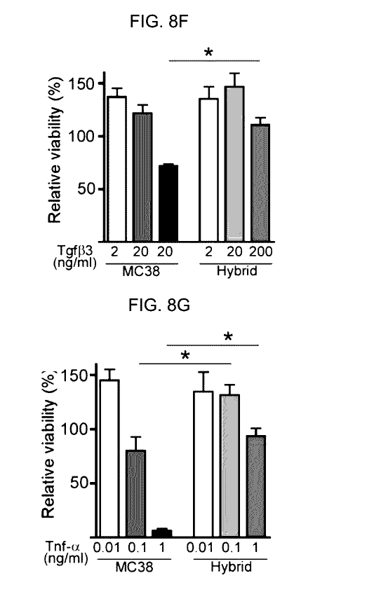

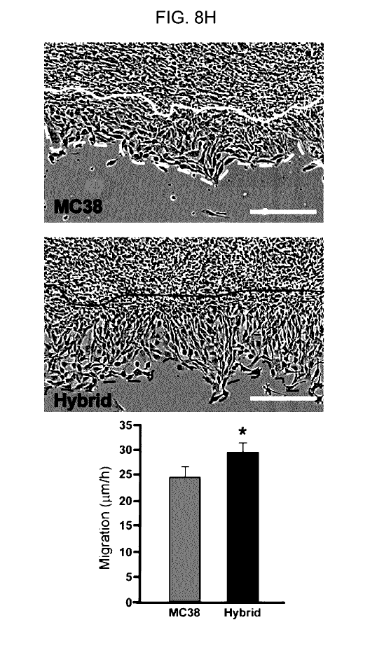

Fusion Hybrids Acquire a Macrophage-associated Phenotype. Despite acquiring a Mφ gene expression profile, Mφ-cancer cell fusion hybrids retained in vitro proliferative capacity similar to unfused cancer cells (FIG. 8A), dividing like cancer cells rather than like Mφs. However, with prolonged growth—past confluence—unfused cancer cells formed cellular aggregates, whereas the MφD-cancer fusion hybrids remained sheet-like, reminiscent of a fibroblast growth pattern (FIG. 8A). This suggested that these hybrids might also have differential growth properties in an in vivo environment. Therefore, in vitro-derived hybrids from MC38 or B16F10 cells were injected into the flank or the dermis, respectively, of immune-competent mice. Indeed, fusion hybrids retained their tumorigenicity. Further, MC38 hybrids grew faster than unfused cancer cells (FIG. 3A), supporting the observation that these cells gained properties for differential growth in a physiologic environment. Further, when MC38-derived MφD-cancer cell fusion hybrids were injected into the spleen, they trafficked to the liver and seeded metastatic foci at greater numbers than unfused cancer cells (FIG. 3B), suggesting that fusion with Mφs provided an in vivo growth advantage. Likewise, B16F10-derived fusions injected retro-orbitally trafficked and grew more abundantly in the lung (FIG. 9B). These findings align with the data identifying the fusion-associated increased expression of GO pathway genes implicated in metastatic spread (FIG. 3C), in particular those pathways that contribute to tumor invasion (attachment, matrix dissolution and migration) as well as pathways involving response to specific microenvironmental cues (Bissell M J & Hines W C, Nat Med 17, 320-329 (2011); Hoshino et al, Cell 134, 215-230 (2008); and Massague, Cell 134, 215-230 (2008); all of which are incorporated by reference herein).

These observations led to testing the impact of different microenvironments on tumor hybrid growth, because the evolving tumor microenvironment provides discrete niches for a context-dependent selective advantage. Therefore, to directly test whether cell fusion altered a cancer cell's ability to respond to microenvironmental interactions, adhesion phenotypes and cytokine-dependent growth responsiveness of MC38-derived fusion hybrids and parental cells on a microenvironment microarray (MEMA) (Lin et al, J Vis Exp doi:10.3791/4152 (2012); incorporated by reference herein) were evaluated. This high throughput assay specifically measures cellular behavior in different microenvironments—extracellular matrix (ECM) and growth factors spotted combinatorially in rows and columns—permitting the comparison of adhesion phenotypes of parental cancer cells, MφDs, and hybrids. Analysis of microenvironment-specific adhesion showed that MC38 cells had a distinct growth factor-independent adhesive preference for discrete ECMs, such as fibronectin (FIG. 8B), and they also displayed enhanced BMP2- and BMP4-specific adhesion to Collagen II and Collagen XXIII (FIG. 8B). Mφs, by contrast, had a higher adhesion to Collagen XXIII and the ECM component vitronectin, and more uniform adhesion across all MEMA conditions relative to parental cancer cells (FIG. 8B). Interestingly, fusion hybrids displayed a combination of adhesion biases, reflecting properties of both parental cells. Further analysis, using hierarchical clustering, distinguished hybrids from parental cancer cells with respect to adhesion on independent microenvironments (FIG. 8B).

To extend these observations, and to more directly test whether Mφ fusion could provide cancer cells with a selective growth advantage, the growth effects of >90 different cytokines and soluble factors on MC38 and hybrid cells were analyzed. A number of growth factors displayed differential influence on MC38 compared to hybrid cells, including Tgfβ1-3, which displayed a clear dose-dependent suppression of MC38 proliferation but had no effect on hybrids (FIGS. 8C, 8E, 8F). Likewise, a moderate, dose-dependent growth-suppressing effect of Hepatic growth factor (Hgf) was apparent on MC38 cells but not on hybrids (FIG. 8D). More strikingly, hybrids were resistant to Tnf-α, which had a profound inhibitory effect on the growth of MC38 cells (FIG. 8G). Resistance of hybrids to cytokine concentrations that suppressed MC38 growth clearly demonstrates the capacity of fusion to influence selectable phenotypes. These results confirm that Mφ fusion can alter cancer cell phenotypes and demonstrate that, under specific growth conditions, spontaneous fusion with Mφ provides cancer cells with a selective growth advantage.

To determine if cell fusion provides a mechanism by which cancer cells acquire Mφ phenotypes, the acquisition of upregulated Mφ genes identified in fusion hybrids was analyzed (FIG. 7D) from key pathways and biologic processes defined by GO terms that are associated with the metastatic process (FIG. 3C). One upregulated gene set identified is involved in modulation of the ECM and includes matrix metalloproteases (Mmp2 and Mmp9) that are capable of degrading type IV collagen, the most abundant component of the basement membrane (Kessenbrock et al, Cell 141, 52-67 (2010); Zeng et al, Carcinogenesis 20, 749-755 (1999); and Sahai, Curr Opin Gene Dev 15, 87-96 (2005); all of which are incorporated by reference herein). Notably, degradation of the basement membrane is an essential step for metastatic invasion by primary tumor cells (Liotta et al, Nature 284, 67-68 (1980); incorporated by reference herein). To determine if Mφ-cancer cell fusion hybrids gain functional Mmp2 activity, Mmp2 gene expression was validated in a number of fusion hybrid lines and measured their functional activity using gelatin zymography (FIG. 3D). Subsets of hybrid cells with high Mmp2 gene expression displayed increased Mmp2 activity relative to their unfused parental cancer cell lines (FIGS. 3D, 9D). Further, consistent with upregulated GO pathways related to cellular migration, in vitro-derived MC38-derived fusion hybrids migrated faster than unfused MC38 cells when analyzed in Mφ cancer cell co-cultures, and in scratch assays comparing MC38-derived fusion hybrids and their parental cell lines (FIGS. 3E, 8F). Together, these data demonstrate that Mφ fusion underlies one mechanism by which a cancer cell can gain functional cell behaviors commonly attributed to a Mφ and related to key behaviors—cell attachment, matrix dissolution and migration—that are associated with cancer cell invasion and metastasis.

GO genes involved in “response to stimulus” that are expressed at high levels in McDs were also upregulated in MφD-cancer fusion hybrids (FIG. 3C). In particular, fusion hybrids harbored elevated expression of the MφD-associated gene colony stimulating factor 1 receptor (Csf1R), which promotes differentiation and function of Mφs (Sherr & Rettenmier, Cancer Surveys 5, 221-232 (1986); incorporated by reference herein)—as well as facilitates metastasis (DeNardo et al, Cancer Discovery 1, 54-67 (2011); incorporated by reference herein). Additionally, hybrids exhibited high expression of Cxcr4, the receptor for the strongly chemotactic lymphocyte cytokine, Sdf1. To determine if acquisition of gene expression translated to a functional migratory response to their ligand, a transwell chemotaxis assay coupled to live-imaging technology (Incucyte Chemotaxis, Essen) was used. Under these conditions, fusion hybrids migrated towards the Csf1 or Sdf1 ligand at different concentrations (shown 25 ng/ml), whereas unfused MC38 cancer cells were incapable of responding to the chemoattractant, and B16F10 cancer cell hybrids had low response (FIGS. 3F, 3G, 9C). Notably, presence of ligand did not change proliferative dynamics of either fusion hybrids or unfused cancer cells; however, incubation with anti-Csf1R or anti-Cxcr4 antibodies prevented the chemotactic response in the fusion hybrid (FIG. 3G). Interestingly, some hybrid lines expressed both Csf1R as well as the Csf1 ligand. Csf1 over-expression in lung cancer has increased tumor cell proliferation and invasion (Hung et al, Lab Invest 94, 371-381 (2014); incorporated by reference herein) and its inhibition correlated with decreased tumor metastasis. Further, aggressive metastatic breast cancer frequently gains Csf1 R expression (Patsialou 2015 supra). How tumor cells gain chemotactically responsive receptor expression is not entirely clear and there may be multiple mechanisms that underlie this change in transcriptional profile.

### Example 4

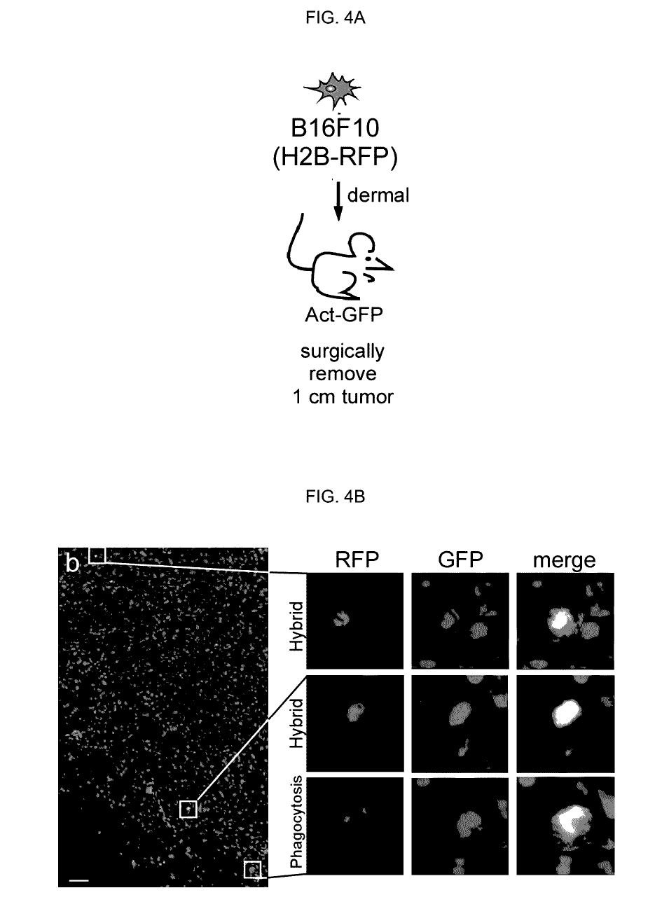

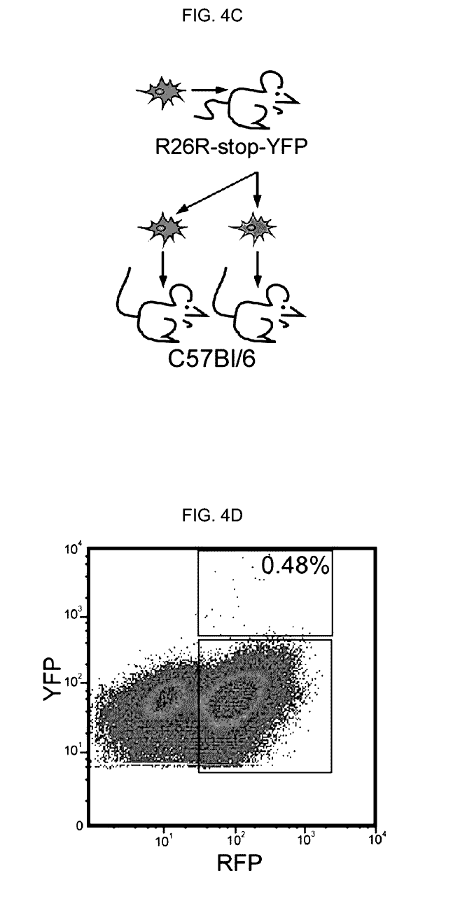

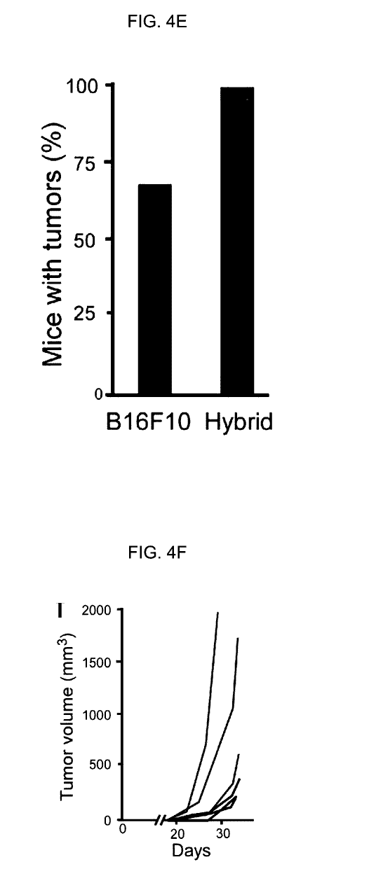

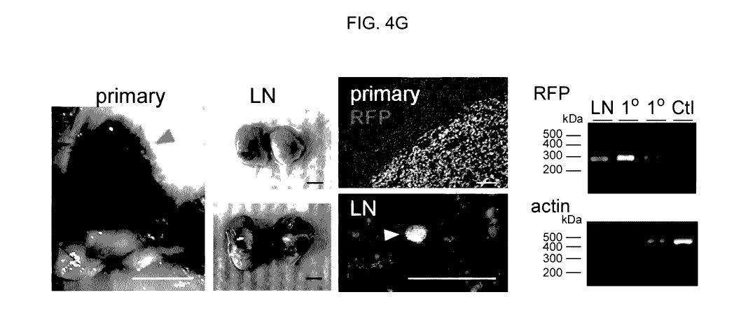

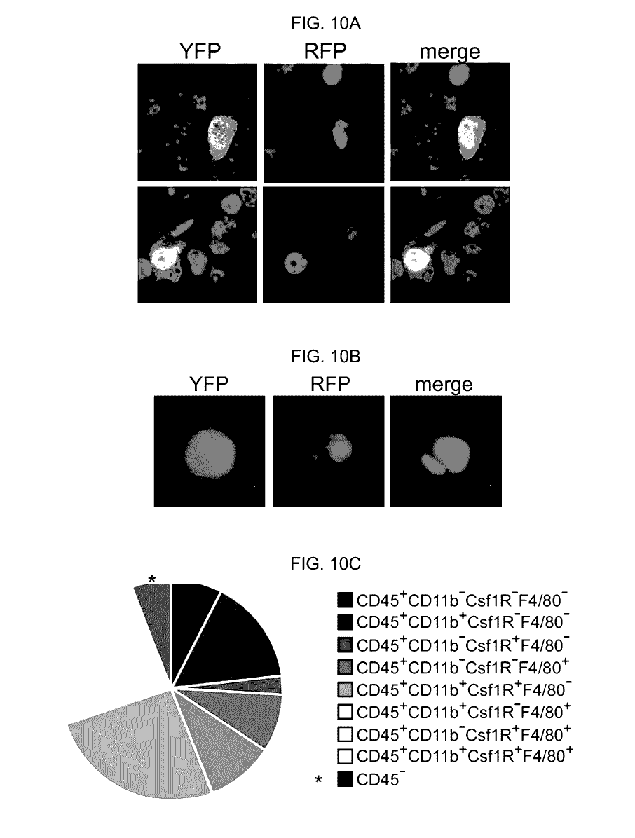

In vivo generation of tumor cell fusion hybrids. While the in vitro-derived fusion hybrids allowed for in-depth functional association of Mφ behaviors and the FISH analysis of human tumors demonstrate that cell fusion occurs in vivo, these studies do not provide insight into the role of fusion hybrids in the metastatic cascade. Therefore, to definitively demonstrate cell fusion in a mouse model of tumorigenesis, MC38 cancer cells were subcutaneously injected into the flank of R26R-YFP Cre reporter mice. In this system, fusion hybrids were identified as RFP⁺YFP⁺ cells; which were detected among unfused tumor cells (RFP⁺) by immunohistochemical analyses of the primary tumor. Orthotopic injection into the cecum of Mφ-MC38 fusion hybrids, however, resulted in pervasive peritoneal seeding and limited the utility of this model. A more tractable system that allowed ease of tumor growth monitoring at an orthotopic site was established—specifically a melanoma model. B16F10 melanoma cells injected into the dermis of a recipient mouse grew into a 1 cm tumor (FIG. 4A). Fluorescence analysis of the primary tumor revealed the presence of RFP⁻/GFP⁺ fusion hybrids (FIG. 4B) and RFP⁺/YFP⁺ fusion hybrids (FIG. 10A). Primary tumor dissociation from B16F10 (H2B-RFP/Cre) cells injected into YFP-reporter mice followed by FACS-isolation (FIG. 10B) and quantification of YFP⁺/RFP⁺ fusion hybrids identified fusion hybrid cells among unfused tumor cells (<0.48%, FIG. 4D). To determine the tumorigenicity and relative growth property of the fusion hybrids, 100 (RFP⁺/YFP⁺) in vivo-derived hybrid cells were reinjected into the dermis of each secondary recipient mice (FIG. 4C), demonstrating that B16F10 hybrid cells retained tumorigenecity (FIG. 4E). Of these injected mice, one mouse injected with B16F10-derived hybrids developed metastatic spread of disease (FIG. 4G). To encourage tumor growth and assess tumor heterogeneity, sufficient numbers of fusion hybrids were collected in order to allow robust tumor growth in additional animals. Surprisingly, the in vivo-derived fusion hybrids appeared to grow more rapidly than the unfused tumor cells (FIG. 4F), but more importantly, the three mice, each injected with 3,000 cells, displayed a level of heterogeneous growth patterns, suggesting that Mφ cell fusion contributes to diverse tumor growth. Collectively, these data indicate that hybrid cells develop spontaneously in vivo, retain tumorigenic capacity, may exhibit accelerated tumor growth and can result in metastasis.

### Example 5

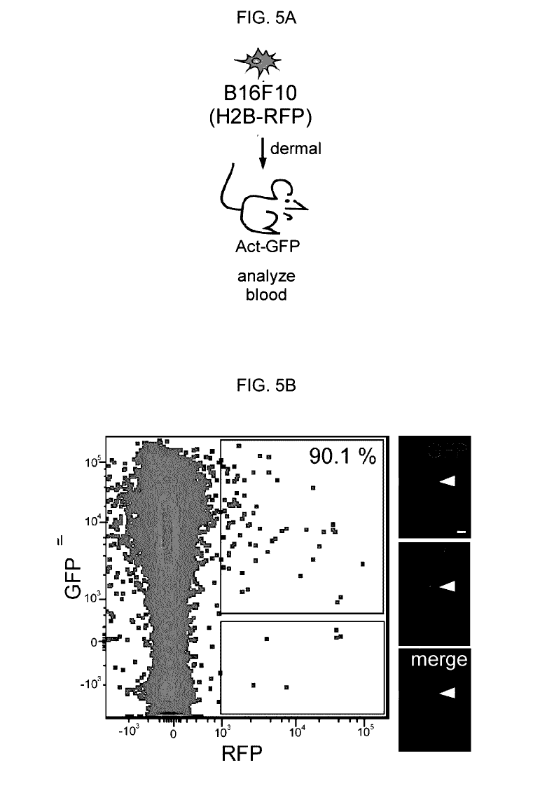

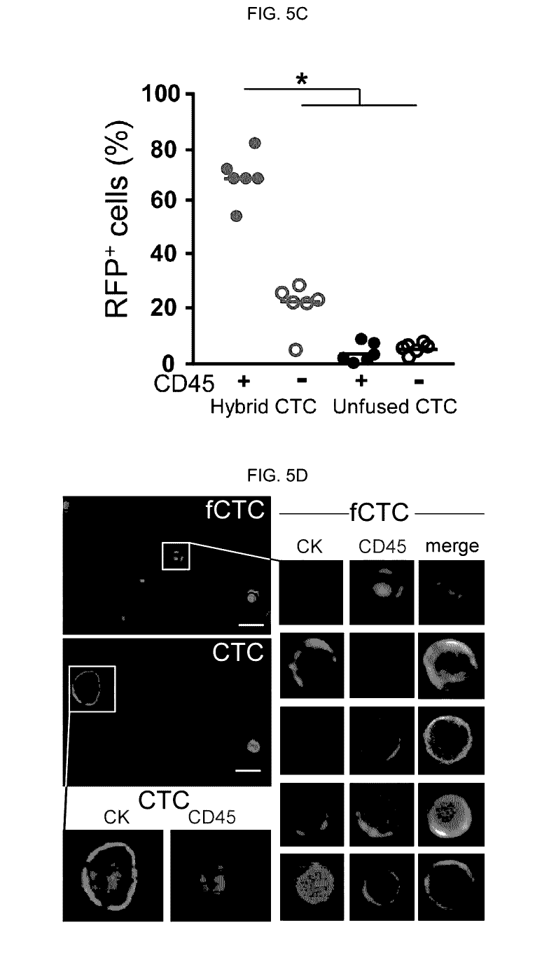

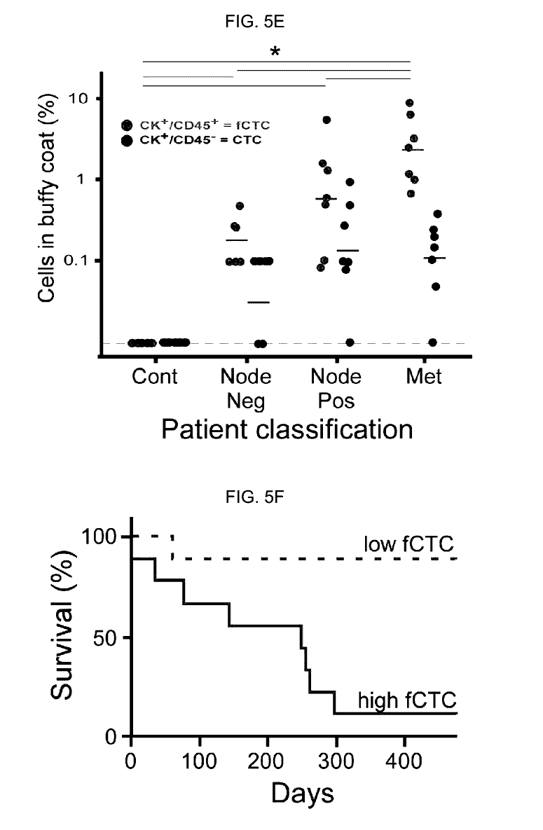

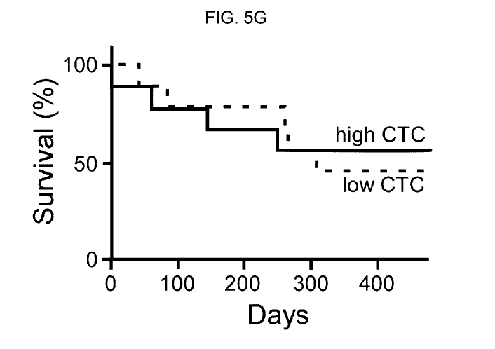

Macrophage-tumor cell fusion hybrids are enriched in circulation. Detectible fusion hybrids in both primary and metastatic sites supported the possibility that fused cancer cells have gained the ability to traffic from the primary tumor to a distant metastatic site. To explore this biologic hallmark of the metastatic cascade, blood was collected from mice with established isogenic tumors (FIG. 5A) in the melanoma model above. Peripheral blood was subjected to flow cytometry for quantification of circulating tumor cells (CTCs). RFP⁺/GFP⁺ fusion hybrids were easily detectible, representing 90.1% of the CTCs, dramatically out-numbering unfused RFP⁺ CTCs (FIG. 5B). Fusion hybrid CTCs were still present in the circulation of tumor free animals, following surgical removal of the primary tumor, suggesting that these hybrid CTCs have long-term survival or that they were seeded by undetectable metastatic foci (data not shown). Imaging of collected individual fused CTCs confirmed their fusion identity and morphologically distinguished them from McDs that had phagocytosed or adhered to a cancer cell (FIG. 5B).

Importantly, the classical definition of CTCs in human cancer is a cell that expresses a tumor antigen (typically Epcam or cytokeratin for epithelial cancers) and does not express the pan-leukocyte antigen CD45 (Fehm et al, Clin Cancer Res 8, 2073-2084 (2002) and Racila et al, Proc Natl Acad Sci USA 95, 4589-4594 (1998); both of which are incorporated by reference herein). McDs normally express CD45. It was therefore hypothesized that MφD-cancer cell fusion hybrids would also express this cell surface epitope and be excluded from conventional CTC isolation. Indeed, the majority of RFP⁺/GFP⁺ fusion hybrids expressed CD45, while unfused RFP⁺ cancer cells largely did not (FIG. 5C). Notably, isolated fusion hybrids displayed a diverse cell surface expression of Mφ antigens (FIG. 10C).

Peripheral blood was collected from human patients with node-negative, node-positive or metastatic pancreatic cancer. /n situ antibody staining (CD45, CK) on isolated leukocytes followed by digital image analyses (FIG. 5D) was performed. This result validated the double-positive expression of CD45 and CK on “fused” CTCs and excluded doublets or clusters of cells. The percentage of fused CTCs expressing CD45⁺/CK⁺ significantly correlated with advanced disease (FIG. 5E) and with overall survival (FIG. 5F. Notably, conventionally defined CTCs (CD45⁻/CK⁺) did not correlate with stage or survival (FIGS. 5E, 5G) and were detected at an order of magnitude lower than fused CTCs in metastatic disease. These data identify a unique, under-appreciated population of tumor cells analogous to the Mφ-cancer cell fusion hybrid cells observed in the mouse models. Significantly, fused CTCs were indicators of disease stage in pancreatic cancer, indicating an avenue for the development of biomarkers for this aggressive disease.

### Example 6

Summary of Examples 1-5. The disclosed in vitro and in vivo data demonstrate that cancer cells fuse spontaneously with primary Mφs. The data indicate that this fusion process influences cancer cell genotypes in a manner that alters their physical behaviors in cellular processes that impact successful navigation along the metastatic cascade. Furthermore, the data demonstrate that cell fusion produces tumorigenic cells that have increased Mφ-associated behaviors, specifically that fusion hybrids express functional levels of the Mφ gene, Csf1R. This finding has important implications for how cell fusions are generated and for how fusion hybrids may respond in the context of chemotherapy or combination treatment with inhibitors to Csf1 R (DeNardo 2011 supra and Ngiow et al, Oncoimmunology 5, e1089381 (2016); incorporated by reference herein).

Also disclosed is evidence that MφD-cancer cell fusion hybrids are differentially modulated by their microenvironment, as specific extracellular conditions provided a selective growth advantage to hybrids but not unfused cancer cells. These discoveries have implications for cancer progression, indicating that Mφ fusion with cancer cells provides a level of tumor cell heterogeneity that allows greater opportunity for positive-selection based on the evolving microenvironment during tumor growth or in response to therapeutic treatment. Thus, Mφ-cancer cell fusion provides a previously unappreciated mechanism by which phenotypic diversity can be achieved within a population of cancer cells, increasing the chances that for any given selection pressure, highly fit subclones will be present within a tumor. Recent evidence strongly supports the occurrence of heterotypic fusion between hematopoietic lineage and cancer cells in humans; and although the frequency of cell fusion in human cancers is unknown, this mechanism has clear potential to drive clonal expansion in the face of specific selective pressures, thereby contributing to the processes of tumor evolution and cancer progression.

These data have now provided an evaluation of Mφ-cancer cell fusion hybrids along the metastatic cascade; most significantly, hybrid cells in peripheral circulation in mouse models of tumor progression and in human patients were identified. Fused CTCs outnumbered unfused, conventionally isolated CTCs in both mice and humans. Notably, the extent of fusion-derived CTCs was highly correlated with tumor stage and overall survival.

It is not currently known whether MφD-cancer cell fusions more efficiently leave the primary tumor site, as the data described here suggest. It is possible that fusion hybrids escape the primary tumor at rates equal to that of unfused tumor cells, but have enhanced survival in the circulation due to their immune heritage promoting immune evasion or other mechanisms. Regardless, these possibilities provide intriguing insights for future examination into immune surveillance of Mφ-tumor cell fusion hybrids, which could impact effectiveness of immune therapy.

Also disclosed is that the acquisition of biologic phenotypes of MφD-tumor cell fusion hybrids is consistent with properties of metastatic tumor cells. This indicates that cell fusion is one mechanism that drives metastatic spread of disease.

### Example 7

Fluorescence in-situ hybridization and immunohistochemical analyses of solid tumors. X- and Y-chromosome FISH probes were hybridized to 5 μm formalin-fixed paraffin embedded primary human tumor sections using CEP X (DXZ1 locus) and Y (DYZ1 locus) probes (Abbott Molecular, Ill.) following manufacturer's protocols. Briefly, tissue was treated with Retrievagen A solutions (BD Biosciences, Calif.), Tissue Digestion Kit II reagents (Kreatech, Netherlands) then hybridized with probe at 80° C. for 5 mins and 37° C. for 12 hr. Tissue sections were permeabilized with graded detergent washes at 24° C., then processed for immunohistochemical staining. Tissue was incubated with antibodies to pan-cytokeratin (Fitzgerald) and counterstained with Hoechst dye (1 μg/mL). Two slides were analyzed for each tumor section. Slides were digitally scanned and quantified by two independent investigators. Areas with Y-chromosome positivity were analyzed by confocal microscopy. Hematoxylin and eosin stain was conducted on adjacent sections.

### Example 8

In situ analyses of human peripheral blood. Patient peripheral blood was collected in heparinized vacutainer tubes (BD), then lymphocytes and peripheral mononuclear cells were isolated using density centrifugation and LeucoSep™ Centrifuge Tubes (Greiner Bio-One) according to manufacturer's protocol. Cells were then adhered to Poly-D-Lysine-coated slides, fixed with 4% paraformaldehyde for 5 min, then stained for CD45 and cytokeratin expression using antibodies to CD45 (eBioscience) and human pan-cytokeratin (Fitzgerald). Tissue was developed with fluorescent-conjugated secondary antibodies (anti-mouse Cy3; Jackson ImmunoResearch and goat anti-guinea pig 488; Invitrogen) then was stained with Hoechst (1 μg/mL). Slides were digitally scanned with a Leica DM6000 B microscope and analyzed using Ariol® software. Manual quantification by three independent investigators of randomly selected regions containing 2,000 cells evaluated CD45 and cytokeratin status of Hoescht+cells. Percentages of fused circulating tumor cells (fCTCs) in the buffy coat correlate with disease stage with significance determined by overall ANOVA post-test, p<6.3×10-8, (p-values: no nodal-met (0.00035), nodal-met (0.05), no nodal-nodal (0.15), while none of the conventional circulating tumor cells (CTC; i.e. CD45⁻) comparisons across stage were statically significant, p-values for no nodal-met (0.31), nodal-met (0.9). Survival analysis was conducted on 18/20 pancreatic patients (two were lost to follow-up) to correlate CTCs with time to death using Kaplan-Meier curve and log rank test using dichotomized biomarkers based on median value. High CK⁺/CD45⁺ (>0.808, median) was associated with a statistically significant increased risk of death (p=0.0029) with a hazard ratio of 8.31, but high CK⁺/CD45⁻ (>0.101, median) did not have a statistically significant effect on time to death (p=0.95).

### Example 9

Mice. All mouse experiments were performed in accordance to the guidelines issued by the Animal Care and Use Committee at Oregon Health & Science University, using approved protocols. Mice were housed in a specific pathogen-free environment under strictly controlled light cycle conditions, fed a standard rodent Lab Chow (#5001 PMI Nutrition International), and provided water ad libitum. The following strains were used in the described studies: C57BL/6J (JAX #000664), Gt(ROSA)26Sortm(EYFP)Cos/J (R26R-stop-YFP; JAX#006148) (Sup.1), Tg(act-EGFP)Y01Osb (Act-GFP; JAX #006567) (Sup.2). Mice of both genders were randomized and analyzed at 8-10 weeks of age. When possible, controls were littermates housed in the same cage as experimental animals.

### Example 10

Cell Culture. MC38 mouse intestinal epithelial cancer cells were kindly provided by Jeffrey Schlom, (NCI, MD) and B16F10 mouse melanoma cells were obtained from the ATCC. Validation of cell lines was confirmed by PCR and by functional metastasis assay for the later. Cell lines, both derived from C57BL/6J mice, were cultured in DMEM+10% serum (Life Technologies, NY). Stable cancer cell lines, MC38(H2B-RFP), MC38(H2B-RFP/Cre, B16F10(H2B-RFP), and B16F10(H2B-RFP/Cre), were generated by retroviral transduction using pBABE-based retroviruses, and polyclonal populations were selected by antibiotic resistance and flow-sorted for bright fluorescence as appropriate. Primary MO derivation was conducted from the bone marrow of R26R-stop-YFP or Act-GFP mice. To elicit MDs, cells were cultured for six days in DMEM+15% serum supplemented with sodium pyruvate, non-essential amino acids (Life Technologies, NY) and 25 ng/ml Csf1 (Peprotech, N.J.).

Cell fusion hybrid generating co-cultures were established in MφD-derivation media without Csf1 for four days. MC38 or B16F10 cells and MDs were co-seeded at a 1:2 ratio at low density. Hybrid cells were FACS-isolated for appropriate fusion markers on a Becton Dickinson InFlux or FACSVantage SE cell sorters (BD Biosciences, Calif.). FACS plots are representative of at least 20 independent MC38 or B16F10 hybrid isolates (technical replicates). Low passage hybrid isolates were established; functional experiments were conducted on passage 8-20 hybrid isolates. Live-imaging of co-cultured cells were performed using an Incucyte Zoom automated microscope system and associated software (Essen Bioscience, Mich.). Technical triplicates generated 36 movies that covered 77.4 mm2 and were screened for hybrid generation and division. Movie represents fusion event captured in one of 21 movies containing hybrids.

### Example 11

EdU-labeling and karyotype analysis. During hybrid generation. Cultured cells were fixed in 4% formaldehyde in PBS and processed for immunohistochemical analyses with antibodies against GFP (1:500; Life Technologies, NY) or RFP (1:1000; Allele Biotechnology, Calif.). 5-ethynyl-2′deoxyuridine-(EdU) labeling and detection was performed according to manufacturer directions (Life Technologies, NY). Briefly, Mφ DNA was labeled with 10 μM EdU supplemented in media for 24 h prior to hybrid generation co-culture. 10 μM EdU was also used for determination of S-phase indices. N=6 biologic and technical replicates were conducted and screened for bi-parental hybrids.

For karyotype analyses. Chromosome spreads from cells in S-phase were prepared using standard protocols, from cells treated for >12 hours with 100 ng/ml Colcemid (Life Technologies, NY) to induce mitotic arrest. DNA was visualized by staining with DAPI; X- and Y-chromosomes were identified using fluorescently labeled nucleotide probes (ID Labs, Canada) as directed by the manufacturer. Images of stained fixed cells and chromosome spreads were acquired using a 40×1.35 UApo oil objective on a DeltaVision-modified inverted microscope (1×70; Olympus) using SoftWorx software (Applied Precision, LLC), and represent maximum intensity projections of deconvolved z-stacks unless otherwise indicated. Experiments were replicated 8 times. Each biologic replicate was analyzed in an independent experiment. A minimum of n=20 cells were analyzed in each experiment. Chromosomes were counted manually by two independent investigators.

### Example 12

Gene expression analysis. Microarray analysis was performed with Mouse 430.2 gene chips (Affymetrix, Calif.) at the OHSU Gene Profiling Shared Resource and data were analyzed using GeneSifter software (Geospiza, Wash.) to identify relative expression differences between cell types (Replicates: Mφ, n=3; MC38, n=3; hybrids, n=5 independent isolates) and produce Gene Ontology analyses. Gene ontology category enrichment was calculated using the GOstats R package (Sup. 3) and visualized using functions from the GOplot R package.

### Example 13

PCR. DNA was extracted from frozen formalin fixed melanoma primary tumor and lymph node sections by 40 min incubation in lysis buffer (25 mM NaOH, 0.2 mM EDTA pH 12) at 95° C. followed by neutralization with equal volumes of neutralization buffer (40 mM Tris-HCl pH 5). RFP primers: fwd 5′-CAGTTCCAGTACGGCTCCAAG-3′ (SEQ ID NO: 1) and rev 5′- CCTCGGGGTACATCCGCTC-3′ (SEQ ID NO: 2). Actin primers: fwd 5′-GAAGTACCCCATTGAACATGGC-3′ (SEQ ID NO: 3) and rev 5′-GACACCGTCCCCAGAATCC-3′ (SEQ ID NO: 4). Reactions were run with a 60° C. annealing temperature.

### Example 14

Microenvironment Arrays. Recombinant proteins (R&D Systems, Minn.) (Millipore, Mass.) were diluted to desired concentrations in print buffer (Arraylt, Calif.) and pair-wise combinations of extracellular matrix proteins and growth factors or cytokines were made in a 384 well plate. A Q-Array Mini microarray printer (Genetix, Calif.) was used to draw from the 384 well plate and print protein combinations onto Nunc 8-well chambered cell culture plates (Thermo Scientific, NY). Each combination was printed in quintuplicate in each array, and arrays were dried at room temperature. Printed MEMAs were blocked for 5 mins using 0.25% w/v F108 copolymer (Sigma-Aldrich, Mo.) in PBS, and then rinsed with PBS and media prior to plating cells. Cells were trypsinized, filtered to exclude cell clumps and counted; 105 cells were plated on each array in 2 ml of DMEM+2.5% serum and incubated for 30 minutes in a humidified tissue culture incubator. Unbound cells were gently removed, and fresh media added; after 12 hours, arrays were fixed with 4% formaldehyde in PBS for 10 mins and stained with DAPI. Adhesion was measured as relative cellular preference: the number of cells occupying a given microenvironment condition relative to the average cell number over all occupied microenvironmental spots across the entire MEMA for each sample. Five replicate samples each for MC38 cells and Mφ, and five independent MC38-derived hybrid isolates were analyzed. Standard two tailed t-tests were performed with p<0.05 reported as significant. Error bars represent S.E.M.

### Example 15

In vitro-derived hybrid proliferation. For phenotypic profiling growth responsiveness to cytokines and soluble factors, 95 different cytokines or soluble signaling molecules were distributed at high, medium and low concentrations in 384 well plates, in 25 μl of RPMI (Life Technologies, NY) supplemented with 1% FBS; and 25 μl of a 1.2×10⁴ cells/ml suspension of hybrid or MC-38 cells in DMEM+4% FBS was added to each well. 99 wells of each plate were left cytokine-free and no cells were added to two of these wells, which served to provide measurements of background signal. Plates were cultured in a humidified incubator for 72 hours, after which 5 ul of MTS reagent was added to each well. Two hours later, absorbance at 490 nm was read with a 384-well plate reader. For each plate, absorbance values for each cytokine-treated well were normalized to the mean absorbance of the cytokine-free wells on that plate, and expressed in terms of standard deviations from the cytokine-free mean. Three independent hybrid isolates and three MC38 replicates were analyzed. Cytokines or factors that showed a potential differential effect on growth of MC38 and hybrid cells were re-tested in 96-well plates. In these experiments, 2.5×104 hybrid or MC38 cells were plated in the presence of three different concentrations for each soluble factor, or in media alone (DMEM+2.5% FBS), in triplicate for each condition. Plates were imaged every two hours for 90 hours, and then cell viability was assessed.

### Example 16

Chemotaxis assay. Chemotaxis assays were performed using IncuCyte™ Chemotaxis Cell Migration Assay (Essen) with at least three technical replicates of triplicate samples. Briefly, 1000 cancer cells were plated in the top wells in DMEM+0.2% FBS after incubation in serum-free media for 20 h. Csf1 or Sdf1 ligand (25 ng/mL) was added to the bottom well and cells were incubated at 37° C. for at least 36 hours with live-imaging. The neutralizing antibodies to the Csf1R (eBioscience), Cxcr4 (Biolegend) and isotype control antibody were added to the top and bottom well (2.5 ng/μL). Migration was quantified by measuring phase contrast area of the top and bottom wells for each timepoint using IncuCyte ZOOM® software. Triplicates of each condition were performed, and the means and standard deviations were calculated. p<0.02 for hybrids treated with Csf1 or Sdf1 relative to hybrids without Csf1 or Sdf1 by unpaired t-test. Two independent hybrid isolates were analyzed. Technical octupulicates (MC38) or sextuplicates (B16F10) with biologic quadruplicates or triplicates were analyzed. For inhibitor studies technical duplicates with biologic triplicates were analyzed.

### Example 17

Scratch Wound Assay. Cells were grown to confluence in 96-well plates and individual scratch wounds were made using an Essen® 96-well WoundMaker™. Wound closure was monitored by live imaging from 2 to 14 hours post scratch and migration rate was determined with IncuCyte ZOOM® software. At least two technical replicates of triplicate samples was performed. p<0.024 by unpaired t-test. Error bars represent s.d.

### Example 18

Migration Analysis. From IncuCyte live imaging of co-cultured Mφs and cancer cells, 24 to 48 h image series containing a cancer-Mφ fusion event was cropped and exported as two separate uncompressed Audio Video Interleave (AVI) files: one containing only the red channel for TrackMate analysis and another containing both red and green channels with a sizing legend. Red channel AVI files were imported into FIJI and converted to 8-bit image series with a mean filter of 1.5 pixels applied. TrackMate analysis was then performed on nuclei with an estimated diameter of 10 pixels and a tolerance of 17.5. Using the Linear Assignment Problem (LAP) Tracker, settings for tracking nuclei were as follows: 75.0 pixel frame to frame linking, 25.0 pixel and 2 frame gap track segment gap closing. Tracks segments were not allowed to split or merge. Using the analysis function in TrackMate, track statistics were exported to an excel file and tracks containing 11 or fewer frames were excluded from analysis. A total of 9 hybrid cells and 536 unfused cells were analyzed with a p<1.1×10-9 by unpaired t-test. Error bars represent s.d.

### Example 19

Gelatin zymography. Cells (MC38-derived hybrids, B16F10-derived hybrids, MC38, B16F10 and Mφs) were cultured in serum free media for 24 hours and cell pellets lysed by sonication. Equivalent protein concentrations from cell lysates were loaded on 10% SDS-polyacrylamide gels co-polymerized with 1 mg/ml of gelatin. Gels were incubated in renaturing solution (2.5% v/v Triton X-100) for 30 min, followed by incubation overnight (16 h) in developing buffer (50 mM Tris-HCl, pH 7.8; 0.2M NaCl; 5 mM CaCl2; 0.02% Brij 35). Gels were stained with Staining solution (0.5% Coomassie blue R-250/5% methanol/10% acetic acid) followed by Destaining solution (10% methanol, 5% acetic acid). Biologic replicates in 5 different technical repeats of 5 different hybrid isolates (MC38) and 2 different isolates (B16F10) were performed.

### Example 20

In vivo analyses of in vitro-derived cell fusion hybrids. For tumor growth, 8-12 week old C57BL/6J mice (Jackson, Me.) were injected with 5×10⁴ cells (MC38, MC38-derived hybrids) or 5×105 cells (B16F10, B16F10-derived hybrids) subcutaneously or intradermally, respectively. Length (L) and width (W) of palpable tumors were measured three times weekly with calipers until tumors reached a maximum diameter of 2 cm. Tumors were surgically removed in survival surgery or animals were sacrificed during tumor removal in accordance with OHSU IACUC guidelines. Animals were observed for at least six months for detection of tumor growth. For each tumor, volume (V) was calculated by the formula V=½(L×W2); volume doubling time for each tumor was extracted from a curve fit to a plot of log tumor volume over time. Curves with R2 values of less than 0.8 were excluded from analysis, as were tumors with six or less dimension measurements; these exclusion criteria were established in response to the unanticipated early ulceration of some tumors, which precluded accurate measurements of length and width, p<0.05, by Mann-Whitney U test. For growth of tumor at metastatic sites, 1×10⁶ MC38 cells were injected into the spleen. Livers were analyzed 3 weeks later for tumor burden by Hematoxylin and Eosin stain. Hybrids formed metastatic foci more readily with a p<0.008 by Mann-Whitney U Test. N=17 (MC38) and n=13 (MC38-derived hybrids) were injected in four different technical replicate experiments. For B16F10 cells, 2.5×10⁵ cells were retro-orbitally injected and lungs were analyzed 16 days post-injection. Melanin marked tumor metastasis were visualized. Duplicate studies of n=3 (B16F10 and B16F10-derived hybrids) were analyzed.

### Example 21

In vivo-derived cell fusion hybrids. For isolation of in vivo-derived hybrids or assessment of circulating tumor cells, 5×10⁵ B16F10 (H2B-RFP with or without Cre) cells were injected intradermally into R26R-YFP or Actin-GFP mice respectively. Once tumors reached 1-2 cm in diameter, it was surgically removed for immunohistochemical analyses or for FACS/flow analyses.

Immunohistochemical analysis of in vivo-derived tumors. B16F10 (H2B-RFP, Cre) primary tumors in Act-GFP or R26R-stop-YFP mice were fixed in 10% buffered formalin, frozen in OCT and 5μm sections were obtained. Tumors from R26R-stop-YFP mice were incubated with antibodies for GFP (1:500; Life Technologies, NY) followed by detection with fluorescent secondary antibody (1:500, Alexa488, Jackson Immuno Research). Nuclei were counterstained with Hoechst (1 μg/mL). Slides were digitally scanned with a Leica DM6000 B microscope and analyzed using Ariol® software. Confocal images were acquired with a FluoView™ FV1000 confocal microscope (Olympus).

FACS-isolation and flow cytometric analyses of fusion hybrids. Tumors were diced, and digested for 30 minutes at 37° C. in DMEM+2 mg/mL Collagenase A (Roche)+DNase (Roche) under stirring conditions. Digested tumor was filtered through a 40 μm filter and washed with PBS. For FACS-isolation, hybrid and unfused cells were isolated by direct fluorescence on a Becton Dickinson InFlux sorter. For flow cytometric analysis, blood was collected retro-orbitally using heparinized micro-hematocrit capillary tubes (Fisher) into K2EDTA-coated tubes (BD). RBC lysis was performed by a 1 minute incubation in 0.2% NaCl followed by addition of the equivalent volume of 1.6% NaCI. Cells were washed and resuspended in FACS Buffer (PBS, 1.0 mM EDTA, 5% FBS). Cells were incubated in PBS containing Live Dead Aqua (1:500, Invitrogen) with Fc Receptor Binding Inhibitor (1:200, eBioscience). Cells were then incubated in FACS buffer for 30 min with CD45⁻PeCy7 (1:8000, Biolegend), CSF1R-BV711 (1:200, Biolegend), F4/80-APC (1:400 Biolegend), CD11b-AF700 (1:200, eBioscience). BD Fortessa FACS machine was used for analyses. Statistical significance of p<2.2×10⁻⁶ by unpaired t-test was determined for CD45⁺ hybrid CTCs relative to CD45⁻ hybrid, CD45⁺ unfused, and CD45⁻ unfused CTCs. Technical duplicates of n=5 or 6 mice were analyzed.

Tumorigenic analyses of FACS-isolated in vivo-derived hybrids. A total of 100 or 3,000 FACS-isolated hybrids and unfused B16F10 cells were reinjected intradermally into C57BL/6J mice. Technical octuplicates were performed. Biologic duplicates, triplicates or quadruplicates were analyzed, dependent upon the number of hybrids isolated from the primary tumor.

### Example 22

Statistical analyses and graphical displays. Dotplots, bar charts and line charts were generated in GraphPad Prism or Excel, which was also used for statistical analyses of these data, including ensuring that data met assumptions of the tests used and comparisons of variance between groups when appropriate. Microscoft Excel was used to perform 2-tailed t-tests. A three-dimensional scatterplot was generated in R using the rgl package. Flow cytometry data were prepared for display using FlowJo software. Microarray gene expression data were displayed as a heatmap prepared using Genesifter software. Heatmap of MEMA data was generated in R using the standard heatmap function and default parameters.

### Example 23

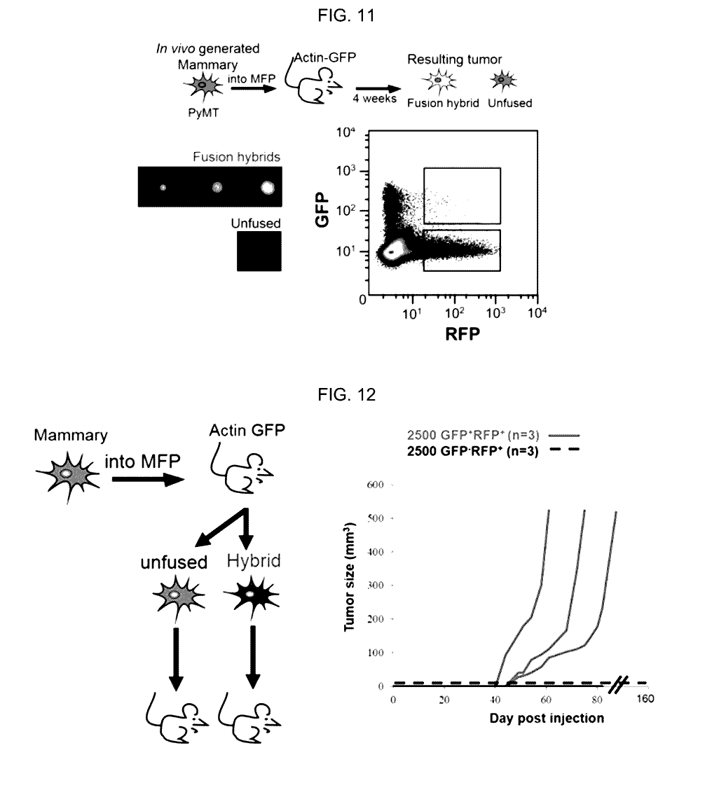

Detection of in vivo derived macrophage/cancer cell fusion hybrids from primary tumor. In vivo-derived blood cell (macrophage) and cancer cell fusion hybrids in a murine model of mammary cancer can be detected and isolated by it dual expression of the tumor marker, RFP and the host blood cell marker GFP (FIG. 11). Fusion-derived hybrids represent a minority of the tumor cells in the primary tumor, however, when injected into secondary recipient mice recapitulate tumorigenesis more readily than unfused cancer cells (FIG. 12). Further, limited dilution studies indicate that fusion hybrids represent a more homogeneous, tumor-initiating cell population (or stem cell-like) when compared to unfused tumor cells (Table 1). This data therefore suggests that cell fusion hybrids harbor greater capacity to seed distant metastatic sites.

TABLE 1
Tumor Growth Analysis from Limited Dilution
Injection of Tumor Cell Populations

| Cells injected | Fusion Hybrid | Unfused |
|---|---|---|
| (number) | Growth | Growth |
| 250,000 | n.d. | growth |
| 25,000 | n.d. | growth |
| 2,500 | growth | no growth |
| 250 | growth | no growth |
| 25 | no growth | no growth |

As will be understood by one of ordinary skill in the art, each embodiment disclosed herein can comprise, consist essentially of or consist of its particular stated element, step, ingredient or component. Thus, the terms “include” or “including” should be interpreted to recite: “comprise or consist of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified.

Unless otherwise indicated, all numbers expressing quantities are to be understood as being modified in all instances by the term “about.” About denotes ±1% of the stated value.

1. A method of identifying a circulating macrophage-tumor cell fusion hybrid from a subject, the method comprising:
contacting a cell obtained from the blood of the subject with a first antibody that binds a macrophage marker, the first antibody comprising a first label and
contacting the cell with a second antibody that binds a tumor antigen, the second antibody comprising a second label, where the second label is distinguishable from the first label;
where a cell with detectable emission of a distinguishable signal from both the first label and the second label is identified as a circulating macrophage-tumor cell fusion hybrid.

2. The method of claim 1 where the first antibody binds CD45 and/or the second antibody binds a cytokeratin.

3. The method of claim 2 where the second antibody comprises a pan-cytokeratin antibody.

4. The method of claim 1 where the first label comprises a first fluorescent label that emits at a first wavelength and the second label comprises a second fluorescent label that emits at a second wavelength and where the first wavelength and the second wavelength can be distinguished.

5. The method of claim 4 further comprising isolating the circulating macrophage tumor cell fusion by fluorescence activated cell sorting (FACS).

6. A method of screening a test compound for inhibition of one or more activities of an in vitro derived macrophage-tumor cell fusion hybrid, the method comprising:
contacting a first sample comprising an in vitro derived macrophage-tumor cell fusion hybrid with the test compound;
contacting a second sample comprising a second in vitro derived macrophage-tumor cell fusion hybrid with a negative control;
measuring one or more of cellular proliferation, cellular migration, or metastasis, in the first sample and the second sample;
wherein a result showing less cellular proliferation, less cellular migration, and/or less metastasis, in the first sample relative to the second sample is an indication that the test compound inhibits the one or more activities of the in vitro derived macrophage-tumor cell fusion hybrid.

7. The method of claim 6 further comprising generating the in vitro derived macrophage-tumor cell fusion hybrid by contacting a macrophage with a tumor cell in a cell culture lacking an agent that promotes cell-cell fusion, where the macrophage expresses a first marker protein and the tumor cell expresses a second marker protein distinguishable from the first marker protein and purifying the macrophage-tumor cell fusion hybrid based on the expression of the first marker protein and the second marker protein.

8. The method of claim 7 where the first marker protein comprises a first fluorescent protein that fluoresces at a first wavelength and where the second marker protein comprises a second fluorescent protein that fluoresces at a second wavelength distinguishable from the first wavelength, and where purifying the macrophage-tumor cell fusion hybrid comprises flow cytometery.

9. The method of claim 8 where the first fluorescent protein comprises RFP, GFP, or YFP and where the second fluorescent protein comprises RFP, GFP, or YFP, provided that the first fluorescent protein and the second fluorescent protein are not both RFP, GFP, or YFP.

10. The method of claim 8 where measuring cellular proliferation comprises performing an MTS assay, where measuring cellular migration comprises performing a chemotaxis assay or a scratch wound assay, or where measuring metastasis comprises measuring liver metastases in an experimental animal after injection of the macrophage-tumor cell fusion hybrid into the spleen of the experimental animal.

11. The method of claim 7 where the macrophage and the tumor cell are each derived from a mouse.

12. The method of claim 11 where the macrophage is derived from a transgenic mouse that expresses a fluorescent protein in macrophages.

13. The method of claim 11 where the tumor cell is derived from a colon adenocarcinoma or a melanoma.

14. The method of claim 13 where the tumor cell comprises the MC38 line or the B16F10 line.

15. A method of screening a test compound for inhibition of proliferation or metastasis of an in vivo derived macrophage-melanoma cell fusion hybrid or macrophage primary mammary tumor cell hybrid, the method comprising:
injecting a melanoma cell line or a primary mammary tumor cell into a first mouse, where the melanoma cell line or primary mammary tumor cell expresses a first fluorescent protein, where the first mouse transgenically expresses a second fluorescent protein distinguishable from the first fluorescent protein, where the melanoma cell line or primary mammary tumor cell forms a tumor in the first mouse, and where the second fluorescent protein is expressed in macrophages;
removing the tumor from the first mouse;
purifying cells that express both the first fluorescent protein and the second fluorescent protein by flow cytometry to obtain in vivo derived macrophage-melanoma cell fusion hybrids or macrophage mammary tumor cell fusion hybrids;
injecting a portion of the purified cells that express both the first fluorescent protein and the second fluorescent protein into a second mouse;
administering the test compound to the second mouse;
injecting a portion of the purified cells that express both the first fluorescent protein and the second fluorescent protein into a third mouse; and
administering a negative control to the third mouse;
where a result showing less proliferation or metastasis of the cells that express both the first fluorescent protein and the second fluorescent protein in the second mouse relative to the third mouse is an indication that the test compound inhibits proliferation or metastasis of the in vivo derived macrophage-melanoma cell fusion hybrid or macrophage mammary tumor cell fusion hybrid.

16. The method of claim 15 where the second mouse and the third mouse are each injected with 50-10,000 cells that express both the first fluorescent protein and the second fluorescent protein.

17. The method of claim 15 comprising injecting the macrophage-melanoma cell fusion hybrids or macrophage-mammary tumor cell hybrids into a first cohort comprising a first plurality of mice and a second cohort comprising a second plurality of mice, injecting the test compound into the first cohort and the negative control into the second cohort.

18. The method of claim 15 comprising removing the tumor from the first mouse when the tumor is 1-2 cm in diameter.

19. The method of claim 15 where the melanoma cell line comprises a B16F10 cell line.

20. The method of claim 15 where the macrophage-melanoma cell fusion hybrids or macrophage mammary tumor cell fusion hybrids are injected into the second mouse, the third mouse, the first plurality of mice and/or the second plurality of mice intradermally.

21. A method of assessing the probability of survival of a human subject with pancreatic cancer past one year, the method comprising:
receiving a peripheral blood sample from the subject, the sample comprising mononuclear cells;
contacting the blood sample with an anti-CD45 antibody, the anti-CD45 antibody comprising a first fluorescent label;
contacting the blood sample with an anti-pan-cytokeratin antibody, the anti-pan cytokeratin antibody comprising a second fluorescent label distinguishable from the first fluorescent label;
contacting the blood sample with Hoescht stain;
counting the CD45+/cytokeratin+/Hoescht+ cells in at least a subset of the cells in the sample;
calculating the percentage of CD45+/cytokeratin+/Hoescht+ cells in the total cells in the subset;
where if more than 0.8% of the cells are CD45+/cytokeratin+/Hoescht+ relative to the total number of cells in the sample, the subject has a less than 12% chance of survival past one year.

22. The method of claim 21 further comprising adhering the peripheral blood sample to a glass substrate and fixing cells from the sample in 4% paraformaldehyde prior to contacting the peripheral blood sample with the anti-CD45 antibody and the anti-pan-cytokeratin antibody.

23. The method of claim 21 where the peripheral blood sample is a buffy coat fraction.

24. The method of claim 21 where the anti-CD45 antibody is a monoclonal antibody derived from the H130 clone and/or the anti-pan cytokeratin antibody is a monoclonal antibody derived from the C1-11 clone or a polyclonal antiserum.

25. The method of claim 21 where the subset of the cells comprises at least 2000 cells.


SEQUENCE LISTING (4 sequences)

SEQ ID NO: 1 - DNA, 21 bp, Artificial Sequence
  RFP primer forward
cagttccagtacggctccaag

SEQ ID NO: 2 - DNA, 19 bp, Artificial Sequence
  RFP primer rev
cctcggggtacatccgctc

SEQ ID NO: 3 - DNA, 22 bp, Artificial Sequence
  Actin primer fwd
gaagtaccccattgaacatggc

SEQ ID NO: 4 - DNA, 19 bp, Artificial Sequence
  Actin primer rev
gacaccgtccccagaatcc